(19) United States
(12) Patent Application Publication
(10) Pub. No.: US 2021/0169922 A1
(43) Pub. Date: Jun. 10, 2021

(54) USE OF POLY-N-ACETYLGLUCOSAMINE NANOFIBERS FOR THE TREATMENT OF GINGIVAL RECESSION

(71) Applicant: Marine Polymer Technologies, Inc., Burlington, MA (US)

(72) Inventors: Marco Finkielsztein, Newton, MA (US); Sergio Finkielsztein, Newton, MA (US)

(73) Assignee: Marine Polymer Technologies, Inc., Burlington, MA (US)

(21) Appl. No.: 17/052,370

(22) PCT Filed: May 10, 2019
(86) PCT No.: PCT/US2019/031689

§ 371 Date: Nov. 2, 2020

Related U.S. Application Data
(60) Provisional application No. 62/670,696, filed on May 11, 2018.

(51) Int. Cl. A61K 31/722; A61K 9/00; A61K 9/70; A61P 1/02; A61K 45/06

(52) U.S. Cl. CPC A61K 31/722; A61K 9/0024; A61K 45/06; A61K 9/0063; A61P 1/02; A61K 9/70

(57) ABSTRACT

In one aspect, described herein are methods for treating gingival recession, or gum recession, comprising administering to a subject in need thereof a composition comprising shortened fibers of poly-N-acetylglucosamine or a derivative thereof (“sNAG nanofibers”). In another aspect, described herein are methods for treating a loss of gingiva tissue, a pocket(s) in the gingiva, or a gingival cleft(s); or a combination of any of the foregoing, comprising administering to a subject in need thereof a composition comprising sNAG nanofibers.

This application claims priority to U.S. provisional application No. 62/670,696, filed on May 11, 2018, which is incorporated herein by reference in its entirety.

### 1. FIELD

In one aspect, described herein are methods for treating gingival recession, or gum recession, comprising administering to a subject in need thereof a composition comprising shortened fibers of poly-N-acetylglucosamine or a derivative thereof (“sNAG nanofibers”). In another aspect, described herein are methods for treating a loss of gingiva tissue, a pocket(s) in the gingiva, or a gingival cleft(s); or a combination of any of the foregoing, comprising administering to a subject in need thereof a composition comprising sNAG nanofibers.

### 2. BACKGROUND

One of the most common conditions of the gingiva is gingival recession. The prevalence of gingival recession increases with age. Gingival recession is “characterized by displacement of gingival margin apically from cement-enamel junction (CEJ) and exposure of root surface to the oral environment.” Bhoomika and Devaraj, 2014, National Journal of Medical Research 4(3): 189. Clinical problems associated with gingival recession include root surface hypersensitivity, root caries, cervical root abrasions, erosions, plaque retention, and aesthetic concern. Id. Current treatments for gingival recession include gingival grafting. There is a need for the treatment of gingival recession by less painful and arduous means.

### 3. SUMMARY

In one aspect, provided herein is a method for treating gingival recession, comprising administering to the gingiva of a subject in need thereof a composition comprising shortened sNAG nanofibers, wherein more than 50% of the sNAG nanofibers are between about 1 to 15 μm in length, and wherein the sNAG nanofibers comprise glucosamine monosaccharides, and wherein at least 70% of the monosaccharides are N-acetylglucosamine monosaccharides. In another aspect, provided herein is a method for treating loss of gingival tissue (e.g., epithelial or connective tissue), pocket formation(s) in the gingiva, or gingival cleft(s), comprising administering to the gingiva of a subject in need thereof a composition comprising shortened sNAG nanofibers, wherein more than 50% of the sNAG nanofibers are between about 1 to 15 μm in length, and wherein the sNAG nanofibers comprise glucosamine monosaccharides, and wherein at least 70% of the monosaccharides are N-acetylglucosamine monosaccharides. In a specific embodiment, the composition is injected into the gingiva. In a particular, embodiment, the composition is injected into the coronal portion of the gingiva.

In one aspect, provided herein is a composition comprising shortened sNAG nanofibers for use in a method for treating gingival recession, wherein the method comprises administering to the gingiva of a subject in need thereof, wherein more than 50% of the sNAG nanofibers are between about 1 to 15 μm in length, and wherein the sNAG nanofibers comprise glucosamine monosaccharides, and wherein at least 70% of the monosaccharides are N-acetylglucosamine monosaccharides. In another aspect, is a composition comprising shortened sNAG nanofibers for use in a method for treating loss of gingival tissue (e.g., epithelial or connective tissue), pocket formation(s) in the gingiva, or gingival cleft(s), wherein the method comprises administering to the gingiva of a subject in need thereof a composition comprising shortened sNAG nanofibers, wherein more than 50% of the sNAG nanofibers are between about 1 to 15 μm in length, and wherein the sNAG nanofibers comprise glucosamine monosaccharides, and wherein at least 70% of the monosaccharides are N-acetylglucosamine monosaccharides. In a specific embodiment, the composition is injected into the gingiva. In a particular, embodiment, the composition is injected into the coronal portion of the gingiva.

In a specific embodiment, a sNAG nanofiber composition described herein is formulated as a suspension or a gel. In a specific embodiment, a sNAG nanofiber composition described herein is injected into the gingiva of the subject using a syringe. In certain embodiments, the syringe is pre-filled with a sNAG nanofiber composition described herein (e.g., a suspension or gel). In some embodiments, the composition comprises 5 to 30 mg of sNAG nanofibers per ml of an isotonic solution (e.g., a saline solution). In certain embodiments, approximately 30 to 40 microliters of such a composition are injected into the gingiva of the subject. In some embodiments, a sNAG nanofiber composition described herein is formulated as a cream, an ointment, a membrane, or a spray. In some embodiment, a sNAG nanofiber composition described herein does not comprise an additional active ingredient.

In some embodiments, a sNAG nanofiber composition described herein is administered to a subject in conjunction with another therapy to treat gingival recession, loss of gingival tissue (e.g., epithelial or connective tissue), pocket formation(s) in the gingiva, or gingival cleft(s). In certain embodiments, a sNAG nanofiber composition described herein is administered to a subject in conjunction with an anti-bacterial agent (e.g., an antibiotic) or anti-viral agent to treat gingival recession, loss of gingival tissue (e.g., epithelial or connective tissue), pocket formation(s) in the gingiva, or gingival cleft(s). In other embodiments, a sNAG nanofiber composition described herein is not administered to a subject in conjunction with an anti-bacterial agent (e.g., an antibiotic) or anti-viral agent to treat gingival recession, loss of gingival tissue (e.g., epithelial or connective tissue), pocket formation(s) in the gingiva, or gingival cleft(s). In some embodiments, a sNAG nanofiber composition described herein is administered to a subject in conjunction with a pain reliever to treat gingival recession, loss of gingival tissue (e.g., epithelial or connective tissue), pocket formation(s) in the gingiva, or gingival cleft(s). In certain embodiments, a sNAG nanofiber composition described herein is administered to a subject in conjunction with an anesthetic to treat gingival recession, loss of gingival tissue (e.g., epithelial or connective tissue), pocket formation(s) in the gingiva, or gingival cleft(s). In some embodiments, a sNAG nanofiber composition described herein is not administered to a subject in conjunction with another therapy to treat gingival recession, loss of gingival tissue (e.g., epithelial or connective tissue), pocket formation(s) in the gingiva, or gingival cleft(s).

In certain embodiments, a sNAG nanofiber composition described herein is administered (e.g., by injection) to the gingiva of a subject only once to treat gingival recession, loss of gingival tissue (e.g., epithelial or connective tissue), pocket formation(s) in the gingiva, or gingival cleft(s). In other embodiments, a sNAG nanofiber composition described herein is administered (e.g., by injection) to the gingiva of a subject on multiple occasions (e.g., the composition is administered on 2 to 6, 2 to 5, 2 to 4, 3 or 2 separate occasions, 2, 3, 4, 5, or 6 separate occasions) to treat gingival recession, loss of gingival tissue (e.g., epithelial or connective tissue), pocket formation(s) in the gingiva, or gingival cleft(s).

In some embodiments, the methods of treatment described herein improve the cement-enamel junction measurement at the mesial, mid or distal section of the coronal gingiva. In certain embodiments, the methods of treatment described herein decrease the reduction in the cement-enamel junction measurement at the mesial, mid or distal section of the coronal gingiva. In some embodiments, the methods of treatment described herein decrease the reduction in the cement-enamel junction measurement at the mesial, mid and distal section of the coronal gingiva. In certain embodiments, the methods of treatment described herein reduce the thinning of the gingiva.

In certain embodiments, the gingival recession, loss of gingival tissue (e.g., epithelial or connective tissue), pocket formation(s) in the gingiva, or gingival cleft(s) treated in accordance with the methods described herein affects incisors. In some embodiments, the gingival recession, loss of gingival tissue (e.g., epithelial or connective tissue), pocket formation(s) in the gingiva, or gingival cleft(s) treated in accordance with the methods described herein affects canines. In certain embodiments, the gingival recession, loss of gingival tissue (e.g., epithelial or connective tissue), pocket formation(s) in the gingiva, or gingival cleft(s) treated in accordance with the methods described herein the gingival recession affects pre-molars. In some embodiments, the gingival recession, loss of gingival tissue (e.g., epithelial or connective tissue), pocket formation(s) in the gingiva, or gingival cleft(s) treated in accordance with the methods described herein is localized. In certain embodiments, the gingival recession, loss of gingival tissue (e.g., epithelial or connective tissue), pocket formation(s) in the gingiva, or gingival cleft(s) treated in accordance with the methods described herein is generalized.

In some embodiments, the gingival recession prior to injection of a composition described herein as measured by cement-enamel junction is −1 mm at the mesial, mid or distal section of the coronal gingiva. In certain embodiments, the gingival recession prior to injection of a composition described herein as measured by cement-enamel junction is −2 mm at the mesial, mid or distal section of the coronal gingiva. In some embodiments, the gingival recession prior to injection of a composition described herein on as measured by cement-enamel junction is −3 mm at the mesial, mid or distal section of the coronal gingiva. In certain embodiments, the gingival recession prior to injection of a composition described herein as measured by cement-enamel junction is −4 mm at the mesial, mid or distal section of the coronal gingiva. In some embodiments, the gingival recession prior to injection of a composition described herein as measured by cement-enamel junction is −5 mm at the mesial, mid or distal section of the coronal gingiva. In certain embodiments, the gingival recession prior to injection of a composition described herein as measured by cement-enamel junction is −6 mm at the mesial, mid or distal section of the coronal gingiva. In some embodiments, the gingival recession prior to injection of a composition described herein as measured by cement-enamel junction is −2 mm to −6 mm at the mesial, mid or distal section of the coronal gingiva.

In certain embodiments, the gingival recession treated in accordance with the methods described herein is classified as class I under Miller's classification system. In some embodiments, the gingival recession treated in accordance with the methods described herein the gingival recession is classified as class II under Miller's classification system. In certain embodiments, the gingival recession treated in accordance with the methods described herein is classified as class III under Miller's classification system. In some embodiments, the gingival recession treated in accordance with the methods described herein is classified as class IV under Miller's classification system.

The subject to be treated using the methods described herein may be a mammal, preferably a human. The subject can also be a livestock animal (e.g., a chicken, a cow, a pig, a goat) or a pet (e.g., a dog or a cat), or any other animal. In a preferred embodiment, the subject is a human subject. In a specific embodiment, the human subject is a human adult. In another specific embodiment, the human subject is an elderly human.

The sNAG nanofibers contemplated in the methods described herein may be of varying lengths, widths and molecular weights as described in Section 5.1, infra. In certain embodiments, the majority (and in certain embodiments, at least or more than 60%, 70%, 80%, 90%, 95% or 99%) of the sNAG nanofibers, or 100% of the sNAG nanofibers, are between about 1 to 15 μm in length. In some embodiments, the majority (and in certain embodiments, at least or more than 60%, 70%, 80%, 90%, 95% or 99%) of the sNAG nanofibers, or 100% of the sNAG nanofibers, are between about 2 to 10 μm, or 4 to 7 μm in length. The sNAG nanofibers of the described length can be obtained, for example, as described below in Section 5.2, infra.

In certain embodiments, more than 50% of the sNAG nanofibers in a composition described herein are between about 2 to 10 μm in length. In some embodiments, the sNAG nanofibers in a composition described herein have an average length of between about 4 to 7 μm in length. In a specific embodiment, 100% of the sNAG nanofibers in a composition described herein are between about 1 to 15 μm in length. In particular, embodiment, the length of the sNAG nanofibers is determined by scanning electron microscopy. In a specific embodiment, the poly-N-acetylglucosamine nanofibers have a β-1→4 configuration.

In certain embodiments, the sNAG nanofibers in a composition described herein were produced by gamma irradiation of poly-N-acetylglucosamine fibers, and wherein the poly-N-acetylglucosamine fibers were irradiated in the form of dried fibers at 500-2,000 kgy, or the poly-β-N-acetylglucosamine fibers were irradiated in the form of wet fibers at 100-500 kgy. In a specific embodiment, the sNAG nanofibers in a composition described herein were produced from a microalgal poly-N-acetylglucosamine.

In certain embodiments, the sNAG nanofibers are derived from microalgae. In another embodiment, the sNAG nanofibers are not derived from crustaceans. In yet another embodiment, the sNAG nanofibers may be derived from microalgae, crustaceans (e.g., shrimp), fungus or any other source.

In certain embodiments, the sNAG nanofibers used in the methods described herein are non-reactive in a biocompatibility test or tests. For example, the sNAG nanofibers used in the methods described herein may be non-reactive when tested in an elution test, an intramuscular implantation test, an intracutaneous test, or a systemic test. In some embodiments, the compositions described herein are non-reactive when tested in an elution test, an intramuscular implantation test, an intracutaneous test, or a systemic test. In other embodiments, the sNAG nanofibers used in the methods described herein have Grade 0 or Grade 1 when tested in an elution test, an intramuscular implantation test, an intracutaneous test, or a systemic test. In yet another embodiment, the sNAG nanofibers used in the methods described herein are at most mildly reactive when tested in an elution test, an intramuscular implantation test, an intracutaneous test, or a systemic test. In one embodiment, the sNAG nanofibers or compositions comprising such nanofibers are non-reactive as determined by an intramuscular implantation test. In certain embodiments, the compositions described herein do not cause an allergenic reaction or an irritation, e.g., at the site of application. In other embodiments, the compositions described herein cause at most a mild allergenic reaction or a mild irritation, e.g., at the site of application.

### 3.1 Terminology

As used herein, the terms “sNAG nanofiber(s)” and “sNAG(s),” are used interchangeably to refer to shortened fibers of poly-N-acetylglucosamine or a derivative(s) thereof. “Taliderm” or “Talymed” is an example of a sNAG nanofiber. See, e.g., Section 5.1, infra, for additional information regarding sNAG nanofibers.

As used herein, the terms “about” and “approximately” mean a range around a given value wherein the resulting value is the same or substantially the same (e.g., within 10%, 5% or 1%) as the expressly recited value. In one embodiment, “about” means within 10% of a given value or range. In another embodiment, the term “about” means within 5% of a given value or range. In another embodiment, the term “about” means within 1% of a given value or range.

As used herein, the term “elderly human” refers to a human 65 years or older.

As used herein, the term “human adult” refers to a human that is 18 years or older.

As used herein, the term “human child” refers to a human that is 1 year to 18 years old.

As used herein, the term “majority” refers to greater than 50%, including, e.g., 50.5%, 51%, 55%, etc.

As used herein, the term “subject” and “patient” are used interchangeably to refer to an animal (e.g., cow, horse, sheep, pig, chicken, turkey, cat, dog, mouse, rat, rabbit, guinea pig, human etc.). In a specific embodiment, the subject is a mammal such as a non-primate or a primate, e.g., a human. In specific embodiments, the subject is a human. See Section 5.5, infra, for more information concerning patients treated in accordance with the methods provided herein.

### 4. BRIEF DESCRIPTION OF FIGURES

FIG. 1. Image shows data collection and measurement procedure. The long arrow represents the connective tissue, while the short arrow represents epithelium tissue above the crest of the bone.

FIG. 2. Average connective tissue thickness in μm: T.M gel (right bar-graph), and PBS only (left bar-graph). * indicates statistical significance.

FIG. 3. Talymed Gel Injections at the Epithelium: Images show the epithelium thickness of the T.M. gel injected gingiva (left), versus PBS injection only (right).

FIG. 4. Talymed Gel Injections at the Connective Tissue: Images show the connective tissue thickness of a T.M. gel injected gingiva (left) versus PBS injection only (right).

FIG. 5A. Effect of irradiation on chemical and physical structure of pGlcNAc fibers. Correlation between molecular weight of pGlcNAc and irradiation level/formulation for irradiation.

FIG. 5B. Effect of irradiation on chemical and physical structure of pGlcNAc fibers. Infrared (IR) spectrum of non-irradiated pGlcNAc slurry (top line), pGlcNAc slurry irradiated at 100 kGy (bottom line), and pGlcNAc slurry irradiated at 200 kGy (middle line).

FIG. 5C. Effect of irradiation on chemical and physical structure of pGlcNAc fibers. Scanning electron microscopic (SEM) analyses of pGlcNAc.

FIG. 5D. Effect of irradiation on chemical and physical structure of pGlcNAc fibers. Scanning electron microscopic (SEM) analyses of sNAG.

### 5. DETAILED DESCRIPTION

Described herein are methods for treating gingival recession, gum recession, loss of gingiva tissue, a pocket(s) in the gingiva, or a gingival cleft(s) comprising administering to a subject in need thereof a composition comprising shortened fibers of poly-N-acetylglucosamine or a derivative thereof (“sNAG nanofibers”).

### 5.1 sNAG Nanofibers

Described herein are sNAG nanofiber compositions. The sNAG nanofibers comprise fibers of poly-N-acetylglucosamine or a derivative(s) thereof, the majority of which are less than 30 microns in length and at least 1 micron in length as determined by any method known to one skilled in the art, for example, by scanning electron microscopy (“SEM”). Such sNAG nanofibers may be obtained, for example, as described herein. See, e.g., Sections 5.2 and 6.3, infra, for methods of making sNAG nanofibers.

In certain embodiments, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers are less than about 30, 25, 20, 15, 12, 10, 9, 8, 7, 6, 5, 4, or 3 microns in length, and at least 1 micron in length as determined by any method known to one skilled in the art, for example, by SEM. In specific embodiments, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers are less than about 20 microns, less than about 15 microns or less than about 10 microns in length, and at least 1 micron in length as determined by any method known to one skilled in the art, for example, by SEM. In specific embodiments, all (100%) of the sNAG nanofibers are between about 1 micron and about 15 microns in length as determined by any method known to one skilled in the art, for example, by SEM. In certain embodiments, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers are equal to or less than 15, 14, 13, 12, 11, 10, 9, 8 or 7 microns in length, and at least 1 micron in length as determined by any method known to one skilled in the art, for example, by SEM. In some embodiments, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers are between 1 to 15, 2 to 15, 3 to 15, 2 to 14, 1 to 12, 2 to 12, 1 to 10, 2 to 10, 3 to 12, 3 to 10, 1 to 9, 2 to 9, 3 to 9, 1 to 8, 2 to 8, 3 to 8, 4 to 8, 1 to 7, 2 to 7, 3 to 7, 4 to 7, 1 to 6, 1 to 5, 1 to 4, or 1 to 3 microns in length as determined by any method known to one skilled in the art, for example, by SEM.

In a specific embodiment, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers are about 8, 7, 6, 5, 4, 3 or 2 microns in length as determined by any method known to one skilled in the art, for example, by SEM. In another specific embodiment, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers are between about 2 to about 10 microns, about 3 to about 8 microns, or about 4 to about 7 microns in length as determined by any method known to one skilled in the art, for example, by SEM. In another specific embodiment, all (100%) of the sNAG nanofibers are between about 1 to about 15 microns, about 3 to about 15 microns, about 2 to about 10 microns, about 3 to about 8 microns, or about 4 to about 7 microns in length as determined by any method known to one skilled in the art, for example, by SEM.

In another embodiment, the average length of the sNAG nanofibers is about 15, 14, 13, 12, 11, 10, 9, 8, 7, 6, 5, 4, 3 or 2 microns as determined by any method known to one skilled in the art, for example, by SEM. In another specific embodiment, the average length of the sNAG nanofibers is between about 2 to about 10 microns, about 3 to about 8 microns, or about 4 to about 7 microns as determined by any method known to one skilled in the art, for example, by SEM. In another specific embodiment, the average length of the sNAG nanofibers is between about 1 to about 15 microns, about 3 to about 15 microns, about 2 to about 10 microns, about 3 to about 8 microns, or about 4 to about 7 microns as determined by any method known to one skilled in the art, for example, by SEM.

In certain embodiments, the sNAG nanofibers are in a range between 0.005 to 5 microns in thickness and/or diameter as determined by electron microscopy. In specific embodiments, the sNAG nanofibers are about 0.01, 0.02, 0.03, 0.04, 0.05, 0.06, 0.07, 0.08, 0.09, 0.1, 0.2, 0.25, 0.3, 0.35, 0.4, 0.45, 0.5, 0.55, 0.6, 0.65, 0.7, 0.75, 0.8, 0.85, 0.9, 1, 1.1, 1.2, 1.3, 1.4, 1.5, 1.6, 1.7, 1.8, 1.9, 2, 2.2, 2.4, 2.6, 2.8, 3 or 4 microns in thickness and/or diameter on average, or any range in between (e.g., 0.02 to 2 microns, 0.02 to 1 microns, 0.02 to 0.75 microns, 0.02 to 0.5 microns, 0.02 to 0.5 microns, 0.05 to 1 microns, 0.05 to 0.75 microns, 0.05 to 0.5 microns, 0.1 to 1 microns, 0.1 to 0.75 microns, 0.1 to 0.5 microns, etc.). In specific embodiments, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers have a thickness or diameter of about 0.02 to 1 microns. In other specific embodiments, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers have a thickness or diameter of about 0.05 to 0.5 microns. In specific embodiments, all (100%) of the sNAG nanofibers have a thickness or diameter of about 0.02 to 1 microns or about 0.05 to 0.5 microns. In certain embodiments, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers have a thickness or diameter of about 0.02 to 2 microns, 0.02 to 1 microns, 0.02 to 0.75 microns, 0.02 to 0.5 microns, 0.02 to 0.5 microns, 0.05 to 1 microns, 0.05 to 0.75 microns, 0.05 to 0.5 microns, 0.1 to 1 microns, 0.1 to 0.75 microns, or 0.1 to 0.5 microns.

In certain embodiments, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers are between about 1 and about 15 microns in length and have a thickness or diameter of about 0.02 to 1 microns.

In certain embodiments, the average molecular weight of the sNAG nanofibers is less than 100 kDa, 90 kDa, 80 kDa, 75 kDa, 70 kDa, 65 kDa, 60 kDa, 55 kDa, 50 kDa, 45 kDa, 40 kDa, 35 kDa, 30 kDa, or 25 kDa. In certain embodiments, the average molecular weight of the sNAG nanofibers is between about 50 kDa to 100 kDa, about 10 kDa to 100 kDa, about 20 kDa to 100 kDa, about 10 kDa to 80 kDa, about 20 kDa to 80 kDa, 20 kDa to 75 kDa, about 25 kDa to about 75 kDa, about 30 kDa to about 80 kDa, about 30 kDa to about 75 kDa, about 40 kDa to about 80 kDa, about 40 kDa to about 75 kDa, about 40 kDa to about 70 kDa, about 40 kDa to about 60 kDa, about 40 kDa to about 55 kDa, about 40 kDa to about 50 kDa, about 50 kDa to about 70 kDa, about 50 kDa to about 80 kDa, about 50 kDa to about 60 kDa, about 45 kDa to about 55 kDa, about 50 kDa to about 55 kDa, or about 55 kDa to about 65 kDa. In certain embodiments, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers have a molecular weight of less than 100 kDa, 90 kDa, 80 kDa, 75 kDa, 70 kDa, 65 kDa, 60 kDa, 55 kDa, 50 kDa, 45 kDa, 40 kDa, 35 kDa, 30 kDa, or 25 kDa. In other embodiments, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers have a molecular weight between about 50 kDa to 100 kDa, about 10 kDa to 100 kDa, about 20 kDa to 100 kDa, about 10 kDa to 80 kDa, about 20 kDa to 80 kDa, 20 kDa to 75 kDa, about 25 kDa to about 75 kDa, about 30 kDa to about 80 kDa, about 30 kDa to about 75 kDa, about 40 kDa to about 80 kDa, about 40 kDa to about 75 kDa, about 40 kDa to about 70 kDa, about 40 kDa to about 60 kDa, about 40 kDa to about 55 kDa, about 40 kDa to about 50 kDa, about 50 kDa to about 70 kDa, about 50 kDa to about 80 kDa, about 50 kDa to about 60 kDa, about 45 kDa to about 55 kDa, about 50 kDa to about 55 kDa, about 50 kDa to about 70 kDa, about 60 kDa to about 70 kDa or about 55 kDa to about 65 kDa. In one embodiment, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers have a molecular weight of about 60 kDa. In one embodiment, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers have a molecular weight of about 50 kDa. In one embodiment, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers have a molecular weight of about 40 kDa. In another embodiment, the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the sNAG nanofibers have a molecular weight of 70,000 daltons±6,000.

In certain embodiments, the sNAG nanofibers described herein have a molecular weight of approximately 60,000 to approximately 80,000 daltons. In a specific embodiment, the sNAG nanofibers described herein have a molecule weight of approximately 60,000 daltons, approximately 65,000 daltons, approximately 70,000 daltons, approximately 75,000 daltons or approximately 80,000 daltons.

In certain embodiments, the sNAG nanofibers in a composition described herein have an average length of between about 1 to 10 μm in length and molecular weight of approximately 50,000 daltons to approximately 100,000 daltons. In certain embodiments, the sNAG nanofibers in a composition described herein have an average length of between about 1 to 10 μm in length and molecular weight of approximately 60,000 daltons to approximately 80,000 daltons. In some embodiments, the sNAG nanofibers in a composition described herein have an average length of between about 1 to 10 μm in length and molecular weight of approximately 50,000 daltons, approximately 55,000 daltons, approximately 60,000 daltons, approximately 65,000 daltons, approximately 70,000 daltons, approximately 75,000 daltons, approximately 80,000 daltons, approximately 85,000 daltons, approximately 90,000 daltons, approximately 95,000 daltons, or approximately 100,000 daltons. In a particular embodiment, the length of the sNAG nanofibers is determined by scanning electron microscopy. In a specific embodiment, the poly-N-acetylglucosamine nanofibers have a β-1→4 configuration.

In certain embodiments, the sNAG nanofibers in a composition described herein have an average length of between about 1 to 8 μm in length and molecular weight of approximately 50,000 daltons to approximately 100,000 daltons. In some embodiments, the sNAG nanofibers in a composition described herein have an average length of between about 4 to 7 μm in length and molecular weight of approximately 50,000 daltons, approximately 55,000 daltons, approximately 60,000 daltons, approximately 65,000 daltons, approximately 70,000 daltons, approximately 75,000 daltons approximately 80,000 daltons, approximately 85,000 daltons, approximately 90,000 daltons, approximately 95,000 daltons, or approximately 100,000 daltons. In certain embodiments, the sNAG nanofibers in a composition described herein have an average length of between about 1 to about 8 μm in length or about 1 to 5 μm in length and molecular weight of 60,000 daltons to 80,000 daltons. In some embodiments, the sNAG nanofibers in a composition described herein have an average length of between about 1 to about 8 μm in length or about 1 to 5 μm in length and molecular weight of 70,000 daltons±6,000 daltons. In a particular embodiment, the length of the sNAG nanofibers is determined by scanning electron microscopy. In a specific embodiment, the poly-N-acetylglucosamine nanofibers have a β-1→4 configuration.

In certain embodiments, 60% to 70%, 60% to 100%, 70% to 100%, 70% to 95%, 70% to 80%, 75% to 80%, 75% to 85%, 85% to 95%, 90% to 95%, 90% to 99% or 95% to 100% of the sNAG nanofibers are acetylated. In some embodiments, 60%, 70%, 75%, 80%, 85%, 90%, 95%, 98%, 99% or 100% of the sNAG nanofibers are acetylated. In other embodiments, more than 70%, 75%, 80%, 85%, 90%, 95%, 98%, 99%, 99.5% or 99.9% of the sNAG nanofibers are acetylated. In some embodiments, equal to or more than 60%, 65%, 70%, 75%, 80%, 85%, 90%, 95% or 99%, or all (100%), of the sNAG nanofibers are acetylated.

In some embodiments, the sNAG nanofibers comprise glucosamine monosaccharides, wherein at least 60%, 65%, 70%, 75%, 80%, 85%, 90%, 95% or 99% of the monosaccharides are N-acetylglucosamine monosaccharides. In other embodiments, the sNAG nanofibers comprise N-acetylglucosamine monosaccharides and glucosamine monosaccharides, wherein at least 60%, 65%, 70%, 75%, 80%, 85%, 90%, 95% or 99% of the monosaccharides are N-acetylglucosamine monosaccharides. In some embodiments, the sNAG nanofibers comprise glucosamine monosaccharide, wherein 60% to 70%, 60% to 100%, 70% to 100%, 70% to 95%, 70% to 80%, 75% to 80%, 75% to 85%, 85% to 95%, 90% to 95%, 90% to 99% or 95% to 100% of the monosaccharides are N-acetylglucosamine monosaccharides. In other embodiments, the sNAG nanofibers comprise N-acetylglucosamine monosaccharide and glucosamine monosaccharides, wherein 60% to 70%, 60% to 100%, 70% to 100%, 70% to 95%, 70% to 80%, 75% to 80%, 75% to 85%, 85% to 95%, 90% to 95%, 90% to 99% or 95% to 100% of the monosaccharides are N-acetylglucosamine monosaccharides.

In certain embodiments, the sNAG nanofibers in a composition described herein have an average length of between about 1 to about 8 μm in length or about 1 to about 5 μm in length and have molecular weight of 60,000 daltons to 80,000 daltons, wherein the sNAG nanofibers comprise glucosamine monosaccharides and wherein at least 70% of the glucosamine monosaccharides are N-acetylglucosamine monosaccharides. In some embodiments, the sNAG nanofibers in a composition described herein have an average length of between about 1 to about 8 μm in length or about 1 to about 5 μm in length and molecular weight of 70,000 daltons 6,000 daltons, wherein the sNAG nanofibers comprises glucosamine monosaccharides and wherein at least 70% of the glucosamine monosaccharides are N-acetylglucosamine monosaccharides. In a particular embodiment, the length of the sNAG nanofibers is determined by scanning electron microscopy. In a specific embodiment, the poly-N-acetylglucosamine nanofibers have a β-1→4 configuration.

In one aspect, the sNAG nanofibers increase the metabolic rate of serum-starved human umbilical cord vein endothelial cells (“EC”) in a MTT assay. A MTT assay is a laboratory test and a standard colorimetric assay (an assay which measures changes in color) for measuring cellular proliferation (cell growth). Briefly, yellow MTT (3-(4,5-Dimethylthiazol-2-yl)-2,5-diphenyltetrazolium bromide, a tetrazole) is reduced to purple formazan in the mitochondria of living cells. This reduction takes place only when mitochondrial reductase enzymes are active, and therefore conversion can be directly related to the number of viable (living) cells. The metabolic rate of cells may be determined by other techniques commonly known to the skilled artisan.

In another aspect, the sNAG nanofibers do not rescue apoptosis of serum-starved EC in a trypan blue exclusion test. A trypan blue exclusion test is a dye exclusion test used to determine the number of viable cells present in a cell suspension. It is based on the principle that live cells possess intact cell membranes that exclude certain dyes, such as trypan blue, Eosin, or propidium, whereas dead cells do not. The viability of cells may be determined by other techniques commonly known to the skilled artisan.

In certain embodiments, compositions comprising the sNAG nanofibers are described, wherein the sNAG nanofibers do one or more of the following: increase the metabolic rate of serum-starved human umbilical cord vein endothelial cells in a MTT assay, or do not rescue apoptosis of serum-starved human umbilical cord vein endothelial cells in a trypan blue exclusion test. In some embodiments, the sNAG nanofibers increase the metabolic rate of serum-starved human umbilical cord vein endothelial cells in a MTT assay and do not rescue apoptosis of serum-starved human umbilical cord vein endothelial cells in a trypan blue exclusion test.

In a specific embodiment, the sNAG nanofibers are biocompatible. Biocompatibility may be determined by a variety of techniques, including, but not limited to such procedures as the elution test, intramuscular implantation, or intracutaneous or systemic injection into animal subjects. Such tests are described in U.S. Pat. No. 6,686,342 (see, e.g., Example 10), which is incorporated by reference herein in its entirety.

In certain embodiments, the sNAG nanofibers used in the methods described herein are non-reactive in a biocompatibility test or tests. For example, the sNAG nanofibers used in the methods described herein may be non-reactive when tested in one, two, or more, or all of the following: an elution test, an intramuscular implantation test, an intracutaneous test, or a systemic test. In other embodiments, the sNAG nanofibers used in the methods described herein have Grade 0 or Grade 1 test score when tested in an elution test, an intramuscular implantation test, an intracutaneous test, or a systemic test. In yet another embodiment, the sNAG nanofibers used in the methods described herein are at most mildly reactive when tested in one, two, or more, or all of the following: an elution test, an intramuscular implantation test, an intracutaneous test, or a systemic test. In certain embodiments, the compositions described herein do not cause an allergenic reaction or an irritation. In other embodiments, the compositions described herein cause at most a mild allergenic reaction or a mild irritation, e.g., at the site of application. The relevant tests and evaluation of test results are described in, e.g., U.S. Pat. Nos. 6,686,342 and 8,858,964, each of which is incorporated herein by reference in its entirety.

In a specific embodiment, the sNAG nanofibers are non-reactive when tested in an intramuscular implantation test. In one aspect, an intramuscular implantation test is an intramuscular implantation test—ISO 4 week implantation, as described in e.g., Section 6.8.3 of U.S. Pat. No. 8,858,964. In certain embodiments, the sNAG nanofibers display no biological reactivity as determined by an elution test (Elution Test Grade=0). In some embodiments, the sNAG nanofibers have a test score equal to “0” and/or are at most a negligible irritant as determined by intracutaneous injection test. In some embodiments, the sNAG nanofibers elicit no intradermal reaction (i.e., Grade I reaction) in Kligman test and/or have a weak allergenic potential as determined by Kligman test.

In certain aspects, the sNAG nanofibers are immunoneutral (i.e., they do not elicit an immune response).

In some embodiments, the sNAG nanofibers are biodegradable. The sNAG nanofibers preferably degrade within about 1 day, 2 days, 3 days, 5 days, 7 days (1 week), 8 days, 10 days, 12 days, 14 days (2 weeks), 17 days, 21 days (3 weeks), 25 days, 28 days (4 weeks), 30 days, 1 month, 35 days, 40 days, 45 days, 50 days, 55 days, 60 days, 2 months, 65 days, 70 days, 75 days, 80 days, 85 days, 90 days, 3 months, 95 days, 100 days or 4 months after administration or implantation into a patient.

In certain embodiments, the sNAG nanofibers do not cause a detectable foreign body reaction. A foreign body reaction, which may occur during wound healing, includes accumulation of exudate at the site of injury, infiltration of inflammatory cells to debride the area, and the formation of granulation tissue. The persistent presence of a foreign body can inhibit full healing. Rather than the resorption and reconstruction that occurs in wound healing, the foreign body reaction is characterized by the formation of foreign body giant cells, encapsulation of the foreign object, and chronic inflammation. Encapsulation refers to the firm, generally avascular collagen shell deposited around a foreign body, effectively isolating it from the host tissues. In one embodiment, treatment of a site (e.g., a wound or a site of a bacterial infection in a wound) with the sNAG nanofibers does not elicit a detectable foreign body reaction in 1 day, 3 days, 5 days, 7 days, 10 days or 14 days after treatment. In one such embodiment, treatment of a site (e.g., a wound) with the sNAG nanofibers does not elicit a foreign body encapsulations in 1 day, 3 days, 5 days, 7 days, 10 days or 14 days after treatment.

In some embodiments, the sNAG nanofibers: (i) comprise fibers, wherein majority of the fibers are between about 1 and 15 microns in length as determined, e.g., by SEM; and (ii) (a) increase the metabolic rate of serum-starved EC in a MTT assay or do not rescue apoptosis of serum-starved EC in a trypan blue exclusion test or both, and (b) are non-reactive when tested in an intramuscular implantation test. In other embodiments, the sNAG nanofibers: (i) comprise fibers, wherein majority of the fibers are between about 3 and 15 microns in length as determined, e.g., by SEM; and (ii) (a) increase the metabolic rate of serum-starved EC in a MTT assay or do not rescue apoptosis of serum-starved EC in a trypan blue exclusion test or both, and (b) are non-reactive when tested in an intramuscular implantation test. In certain embodiments, the sNAG nanofibers: (i) comprise fibers, wherein majority of the fibers are between about 1 and 12 microns in length as determined, e.g., by SEM; and (ii) (a) increase the metabolic rate of serum-starved EC in a MTT assay or do not rescue apoptosis of serum-starved EC in a trypan blue exclusion test or both, and (b) are non-reactive when tested in an intramuscular implantation test. In certain embodiments, the sNAG nanofibers: (i) comprise fibers, wherein majority of the fibers are between about 4 and 7 microns in length as determined, e.g., by SEM; and (ii) (a) increase the metabolic rate of serum-starved EC in a MTT assay or do not rescue apoptosis of serum-starved EC in a trypan blue exclusion test or both, and (b) are non-reactive when tested in an intramuscular implantation test.

In a specific embodiment, the sNAG nanofibers are obtained by irradiating poly-N-acetylglucosamine or a derivative thereof. See Section 5.1.1, infra, regarding poly-N-acetylglucosamine or derivatives thereof and Section 5.2 and 6.3, infra, regarding methods for producing the sNAG nanofibers using irradiation. Irradiation may be used to reduce the length of poly-N-acetylglucosamine fibers (e.g., poly-β-1→4-N-acetylglucosamine) or poly-N-acetylglucosamine derivative fibers to form shortened poly-N-acetylglucosamine fibers or shortened poly-N-acetylglucosamine derivative fibers, i.e. sNAG nanofibers. Specifically, irradiation may be used to reduce the length and molecular weight of poly-N-acetylglucosamine or a derivative thereof without disturbing its microstructure. The infrared spectrum (IR) of sNAG nanofibers is similar to, about the same as, or equivalent to that of the non-irradiated poly-N-acetylglucosamine or a derivative thereof. In some embodiments, the IR spectrum of the sNAG nanofibers is not statistically different than the IR spectrum of the non-irradiated poly-N-acetylglucosamine or a derivative thereof. See, e.g., Section 6.3, infra, regarding the IR spectrum and maintenance of the microstructure of non-irradiated poly-N-acetylglucosamine (e.g., poly-β-1→4-N-acetylglucosamine). In a specific embodiment, the sNAG nanofibers have an IR spectrum that is the same, similar to, or not statistically different than the IR spectrum of fibers of poly-N-acetylglucosamine (e.g., poly-β-1→4-N-acetylglucosamine) with fiber dimensions averaging 20-50 nm×1-2 nm×100 μm and the sNAG nanofibers maintain the microstructure of fibers of poly-N-acetylglucosamine (e.g., poly-β-1→4-N-acetylglucosamine) with fiber dimensions averaging 20-50 nm×1-2 nm×100 μm. In some embodiments, the sNAG nanofibers have a β-1→4 poly-N-acetylglucosamine configuration. In other embodiments, the sNAG nanofibers have a α-1→4 poly-N-acetylglucosamine configuration.

In one embodiment, the sNAG nanofibers are not derived from chitin or chitosan. Whereas in another embodiment, the compositions described herein may be derived from chitin or chitosan, or the sNAG nanofibers may be derived from chitin or chitosan. In a specific embodiment, the sNAG nanofibers are derived from microalgae.

5.1.1 Poly-N-Acetylglucosamine and Derivatives Thereof

U.S. Pat. Nos. 5,622,834; 5,623,064; 5,624,679; 5,686,115; 5,858,350; 6,599,720; 6,686,342; 7,115,588 and U.S. Patent Pub. 2009/0117175 (each of which is incorporated herein by reference) describe the poly-N-acetylglucosamine and derivatives thereof, and methods of producing the same. In some embodiments, the poly-N-acetylglucosamine has a β-1→4 configuration. In other embodiments, the poly-N-acetylglucosamine has a α-1→4 configuration. In some embodiments, the poly-N-acetylglucosamine and derivatives thereof is in the form of a polymer. In some embodiments, the polymer is in the form of a fiber. In preferred embodiments, the poly-N-acetylglucosamine and derivatives thereof is in the form of a fiber.

Poly-N-acetylglucosamine can, for example, be produced by, and may be purified from, microalgae, preferably diatoms. The diatoms which may be used as starting sources for the production of the poly-N-acetylglucosamine include, but are not limited to members of the Coscinodiscus genus, the Cyclotella genus, and the Thalassiosira genus. Poly-N-acetylglucosamine may be obtained from diatom cultures via a number of different methods, including the mechanical force method and chemical/biological method known in the art (see, e.g., U.S. Pat. Nos. 5,622,834; 5,623,064; 5,624,679; 5,686,115; 5,858,350; 6,599,720; 6,686,342; and 7,115,588, each of which is incorporated herein by reference in its entirety). In certain embodiments, the poly-N-acetylglucosamine is not derived from one or more of the following: a shell fish, a crustacean, an insect, a fungi or yeasts.

In one embodiment, poly-β-1→4-N-acetylglucosamine is derived from a process comprising a) treating a microalgae comprising a cell body and a poly-β-1→4-N-acetylglucosamine polymer fiber with a biological agent (such as hydrofluoric) capable of separating the N-acetylglucosamine polymer fiber from the cell body for a sufficient time so that the poly-β-1→4-N-acetylglucosamine polymer fiber is released from the cell body; b) segregating the poly-β-1→4-N-acetylglucosamine polymer fiber from the cell body; and c) removing contaminants from the segregated poly-β-1→4-N-acetylglucosamine polymer fiber, so that the poly-β-1→4-N-acetylglucosamine polymer is isolated and purified.

In other embodiments, the poly-β-1→4-N-acetylglucosamine may be derived from one or more of the following: a shell fish, a crustacean, an insect, a fungi or yeasts. In certain embodiments, the compositions described herein do not comprise chitin or chitosan.

In certain embodiments, a poly-N-acetylglucosamine composition comprises 60% to 70%, 60% to 100%, 70% to 100%, 70% to 95%, 70% to 80%, 75% to 80%, 75% to 85%, 85% to 95%, 90% to 95%, 90% to 99% or 95% to 100% of acetylated glucosamine (i.e., N-acetylglucosamine) monosaccharides. In some embodiments, a poly-N-acetylglucosamine composition comprises 60%, 70%, 75%, 80%, 85%, 90%, 95%, 98%, 99% or 100% of acetylated glucosamine (i.e., N-acetylglucosamine) monosaccharides. In other embodiments, a poly-N-acetylglucosamine composition comprises more than 60%, 70%, 75%, 80%, 85%, 90%, 95%, 98%, 99%, 99.5% or 99.9% of the acetylated glucosamine. In some embodiments, a poly-N-acetylglucosamine composition comprises equal to or more than 60%, 65%, 70%, 75%, 80%, 85%, 90%, 95% or 99%, or all (100%), of the acetylated glucosamine.

In some embodiments, a poly-N-acetylglucosamine composition comprises glucosamine monosaccharides, wherein at least 60%, 65%, 70%, 75%, 80%, 85%, 90%, 95% or 99% of the monosaccharides are N-acetylglucosamine monosaccharides. In other embodiments, a poly-N-acetylglucosamine composition comprises N-acetylglucosamine monosaccharides and glucosamine monosaccharides, wherein at least 60%, 65%, 70%, 75%, 80%, 85%, 90%, 95% or 99% of the monosaccharides are N-acetylglucosamine monosaccharides. In some embodiments, a poly-N-acetylglucosamine composition comprises glucosamine monosaccharides, wherein 60% to 70%, 60% to 100%, 70% to 100%, 70% to 95%, 70% to 80%, 75% to 80%, 75% to 85%, 85% to 95%, 90% to 95%, 90% to 99% or 95% to 100% of the monosaccharides are N-acetylglucosamine monosaccharides. In other embodiments, a poly-N-acetylglucosamine composition comprises N-acetylglucosamine monosaccharides and glucosamine monosaccharides, wherein 60% to 70%, 60% to 100%, 70% to 100%, 70% to 95%, 70% to 80%, 75% to 80%, 75% to 85%, 85% to 95%, 90% to 95%, 90% to 99% or 95% to 100% of the monosaccharides are N-acetylglucosamine monosaccharides.

Derivatives of poly-N-acetylglucosamine may also be used in a composition described herein. Derivatives of poly-N-acetylglucosamine and methods of making such derivatives are described in U.S. Pat. No. 5,623,064 (see, e.g., Section 5.4), which is incorporated by reference herein in its entirety. Poly-N-acetylglucosamine may be derivatized by being sulfated, phosphorylated and/or nitrated. Poly-N-acetylglucosamine derivatives include, e.g., sulfated poly-N-acetylglucosamine derivatives, phosphorylated poly-N-acetylglucosamine derivatives, or nitrated poly-N-acetylglucosamine derivatives. Additionally, one or more of the monosaccharide units of the poly-N-acetylglucosamine may contain one or more sulfonyl groups one or more O-acyl groups. One or more of the monosaccharides of the poly-N-acetylglucosamine, may contain an O-alkyl group. One or more of the monosaccharide units of the poly-N-acetylglucosamine may be an alkali derivative. In one embodiment, the poly-N-acetylglucosamine is derivatized with lactic acid. Wherein, in another embodiment, the derivative is not derivatized with lactic acid.

In a specific embodiment, derivatives of poly-N-acetylglucosamine are not used to produce sNAG nanofibers. In some embodiments, a derivative of poly-N-acetylglucosamine is low molecular weight polymer, which is soluble in low pH. In certain embodiments, a derivative of poly-N-acetylglucosamine is a small particle (1 to 10 μm)/low molecular weight polymer that is insoluble in low pH.

### 5.2 Methods of Making sNAG Nanofibers

The poly-N-acetylglucosamine fibers, and any derivatives of poly-N-acetylglucosamine fibers described above, can be irradiated as dry fibers or fiber membranes. Alternatively, poly-N-acetylglucosamine fibers, and any derivatives of poly-N-acetylglucosamine fibers described above, can be irradiated when wet. The methods of making sNAG nanofibers by irradiation and the sNAG nanofibers so produced have been described in U.S. Patent Pub. No. 2009/0117175, and U.S. Pat. Nos. 8,871,247, 9,139,663, and 9,139,664, each of which is incorporated by reference herein in its entirety.

In certain embodiments, the poly-N-acetylglucosamine fibers are formulated into a suspension/slurry or wet cake for irradiation. Irradiation can be performed prior to, concurrently with or following the formulation of the fibers into its final formulation, such as a dressing. Generally, the fiber content of suspensions/slurries and wet cakes can vary, for example from about 0.5 mg to about 50 mg of fiber per 1 ml of distilled water are used for slurries and from about 50 mg to about 1000 mg of fiber per 1 ml of distilled water are used for wet cake formulations. The fiber may first be lyophilized, frozen in liquid nitrogen, and pulverized, to make it more susceptible to forming a suspension/slurry or wet cake. Also, the suspensions/slurries can be filtered to remove water such that a wet cake is formed. In certain aspects, the fiber is irradiated as a suspension comprising about 0.5 mg, 1 mg, 2 mg, 3 mg, 4 mg, 5 mg, 6 mg, 7 mg, 8 mg, 9 mg, 10 mg, 12 mg, 15 mg, 18 mg, 20 mg, 25 mg or 50 mg of polymer or fiber per ml of distilled water, or any range in between the foregoing embodiments (e.g., 1-10 mg/ml, 5-15 mg/ml, 2-8 mg/ml, 20-50 mg/ml, etc.). In other aspects, the fiber is irradiated as a wet cake, comprising about 50-1,000 mg fiber per 1 ml of distilled water. In specific embodiments, the wet cake comprises about 50, 100, 200, 300, 400, 500, 600, 700, 800, 900 or 1000 mg of fiber per 1 ml distilled water, or any range in between (e.g., 100-500 mg/ml, 300-600 mg/ml, 50-1000 mg/ml, etc.).

The irradiation is preferably in the form of gamma radiation, e-beam radiation, or x-rays. Two sources of irradiation are preferred: radioactive nuclides and electricity. In specific embodiment, the radioactive nuclides are cobalt-60 and cesium-137. Both of these nuclides emit gamma rays, which are photons containing no mass. The gamma rays have energies from 0.66 to 1.3 MeV. Using electricity, electrons are generated and accelerated to energies up to 10 MeV or higher. When irradiating fibers to reduce their size, a consideration to take into account is that the depth of penetration of materials with densities similar to water by 10 MeV electrons is limited to about 3.7 cm with one-sided exposure or about 8.6 cm with two-sided exposure. Depth of penetration decreases at lower electron energies. Electron energy can be converted to x-rays by placing a metal (usually tungsten or tantalum) target in the electron beam path. Conversion to x-rays is limited to electrons with energies up to 5 MeV. X-rays are photons with no mass and can penetrate fibers similar to gamma rays. There is only about 8% efficiency in the conversion of electron energy to x-ray energy. High powered electron beam machines are needed in x-ray production facilities to account for the low conversion efficiency.

In a specific embodiment, the irradiation is gamma irradiation.

The absorbed dose of radiation is the energy absorbed per unit weight of product, measured in gray (gy) or kilogray (kgy). For dried fibers, the preferred absorbed dose is about 500-2,000 kgy of radiation, most preferably about 750-1,250 kgy or about 900-1,100 kgy of radiation. For wet fibers, the preferred absorbed dose is about 100-500 kgy of radiation, most preferably about 150-250 kgy or about 200-250 kgy of radiation.

The dose of radiation can be described in terms of its effect on the length of the fibers. In specific embodiments, the dose of radiation used preferably reduces the length of the fiber by anywhere from about 10% to 90% of the starting length of the fiber, respectively. In specific embodiments, the average length is reduced by about 10%, by about 20%, by about 30%, by about 40%, by about 50%, by about 60%, by about 70%, by about 80%, or by about 90%, or any range in between (e.g., 20-40%, 30-70%, and so on and so forth). Alternatively, the dose of radiation used preferably reduces the length of the fiber to anywhere from 1 to 30 microns. In specific embodiments, and depending on the starting fiber length, the average length of the fiber is reduced to less than about 20 microns, less than about 15 microns, less than about 14 microns, less than about 13 microns, less than about 12 microns, less than about 11 microns, less than about 10 microns, less than about 8 microns, less than about 7 microns, less than about 5 microns, less than about 4 microns, less than about 3 microns, less than 2 microns, or less than 1 microns. In certain embodiments, the length of the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the fibers is reduced to no greater than about 20 microns, no greater than about 15 microns, no greater than about 12 microns, no greater than about 10 microns, no greater than about 8 microns, no greater than about 7 microns, or no greater than about 5 microns. In certain embodiments, irradiation of the fibers reduces the length of the majority (and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the fibers to anywhere between about 1 to 20 microns, between about 1 to 15 microns, between about 2 to 15 microns, between about 3 to 15 microns, between about 1 to 12 microns, between about 2 to 12 microns, between about 1 to 10 microns, between about 2 to 10 microns, between about 1 to 8 microns, between about 2 to 8 microns, between about 1 to 7 microns, between about 2 to 7 microns, between about 3 to 8 microns, between about 4 to 7 microns, between about 1 to 5 microns, between about 2 to 5 microns, between about 3 to 5 microns, between about 4 to 10 microns, or any ranges between the foregoing lengths, which are also encompassed.

In certain embodiments, the average length of the fibers is reduced to no greater than about 20 microns, no greater than about 15 microns, no greater than about 12 microns, no greater than about 10 microns, no greater than about 8 microns, no greater than about 7 microns, or no greater than about 5 microns. In other embodiments, the average length of the fibers is reduced to no less than 1 micron. In certain embodiments, irradiation of the fibers reduces the average length of the fibers to anywhere between about 1 to 20 microns, between about 1 to 15 microns, between about 2 to 15 microns, between about 3 to 15 microns, between about 1 to 12 microns, between about 2 to 12 microns, between about 1 to 10 microns, between about 2 to 10 microns, between about 1 to 8 microns, between about 2 to 8 microns, between about 1 to 7 microns, between about 2 to 7 microns, between about 3 to 8 microns, between about 4 to 7 microns, between about 1 to 5 microns, between about 2 to 5 microns, between about 3 to 5 microns, between about 4 to 10 microns, or any ranges between the foregoing lengths, which are also encompassed. In a specific embodiment, the length of the fibers is determined by SEM.

In certain embodiments, the length of the fibers is reduced to no greater than about 20 microns, no greater than about 15 microns, no greater than about 12 microns, no greater than about 10 microns, no greater than about 8 microns, no greater than about 7 microns, or no greater than about 5 microns, as determined by any method known to one skilled in the art, for example, by SEM. In other embodiments, the length of the fibers is reduced to no less than 1 micron, as determined by any method known to one skilled in the art, for example, by SEM. In certain embodiments, irradiation of the fibers reduces the length of the fibers to anywhere between about 1 to 20 microns, between about 1 to 15 microns, between about 2 to 15 microns, between about 3 to 15 microns, between about 1 to 12 microns, between about 2 to 12 microns, between about 1 to 10 microns, between about 2 to 10 microns, between about 1 to 8 microns, between about 2 to 8 microns, between about 1 to 7 microns, between about 2 to 7 microns, between about 3 to 8 microns, between about 4 to 7 microns, between about 1 to 5 microns, between about 2 to 5 microns, between about 3 to 5 microns, between about 4 to 10 microns, or any ranges between the foregoing lengths, which are also encompassed, as determined by any method known to one skilled in the art, for example, by SEM.

The dose of radiation can also be described in terms of its effect on the molecular weight of the fiber. In specific embodiments, the dose of radiation used preferably reduces the molecular weight of the fiber by anywhere from about 10% to 90% of the starting weight of the fiber. In specific embodiments, the average molecular weight is reduced by about 10%, by about 20%, by about 30%, by about 40%, by about 50%, by about 60%, by about 70%, by about 80%, or by about 90%, or any range in between (e.g., 20-40%, 30-70%, and so on and so forth). Alternatively, the dose of radiation used preferably reduces the molecular weight of the fiber to anywhere from 1,000 to 1,000,000 daltons. In specific embodiments, and depending on the starting molecular weight, the average molecular weight of the fiber is reduced to less than 1,000,000 daltons, less than 750,000 daltons, less than 500,000 daltons, less than 300,000 daltons, less than 200,000 daltons, less than 100,000 daltons, less than 90,000 daltons, less than 80,000 daltons, less than 70,000 daltons, less than 60,000 daltons, less than 50,000 daltons, less than 40,000 daltons, less than 25,000 daltons, less than 10,000 daltons, or less than 5,000 daltons. In certain embodiments, the average molecular weight is reduced to no less than 500 daltons, no less than 1,000 daltons, no less than 2,000 daltons, no less 3,500 daltons, no less than 5,000 daltons, no less than 7,500 daltons, no less than 10,000 daltons, no less than 25,000 daltons, no less than 40,000 daltons, no less than 50,000 daltons, no less than 60,000 daltons or no less than 100,000 daltons. Any ranges between the foregoing average molecular weights are also encompassed; for example, in certain embodiments, irradiation of the fiber reduces the average molecular weight to anywhere between 10,000 to 100,000 daltons, between 1,000 and 25,000 daltons, between 50,000 and 500,000 daltons, between 25,000 and 100,000 daltons, between 30,000 and 90,000 daltons, between about 40,000 and 80,000 daltons, between about 40,000 and 60,000 daltons, between about 25,000 and 75,000 daltons, between 50,000 and 80,000 daltons, between about 50,000 and 70,000 daltons, or between about 55,000 and 65,000 daltons and so on and so forth. In certain embodiments, irradiation of the fibers reduces the molecular weight of the majority and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the fibers to anywhere between about 20,000 and 100,000 daltons, about 25,000 and 75,000 daltons, about 30,000 and 90,000 daltons, about 40,000 and 80,000 daltons, about 50,000 and 70,000 daltons, about 40,000 and 60,000 daltons, or about 55,000 and 65,000 daltons. In certain embodiments, irradiation of the fibers reduces the molecular weight of the majority and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the fibers to about 60,000 daltons. In certain embodiments, irradiation of the fibers reduces the molecular weight of the majority and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the fibers to about 50,000 daltons. In certain embodiments, irradiation of the fibers reduces the molecular weight of the majority and in certain embodiments, at least 60%, 70%, 80%, 90%, 95%, 98%, 99%, 99.5%, 99.8%, 99.9%, or 100%, or between 55% to 65%, 55% to 75%, 65% to 75%, 75% to 85%, 75% to 90%, 80% to 95%, 90% to 95%, or 95% to 99%) of the fibers to about 40,000 daltons.

Following irradiation, slurries can be filtered and dried, and wet cakes can be dried, to form compositions that are useful in the treatment of gingival recession, a loss of gingiva tissue, a pocket(s) in the gingiva, or a gingiva cleft(s).

### 5.3 Compositions Comprising sNAG Nanofibers

A composition comprising the sNAG nanofibers may be formulated as a cream, a membrane, a film, a liquid solution, a suspension, a powder, a paste, an ointment, a gelatinous composition, an aerosol, a gel, or a spray. In one embodiment, a composition comprising the sNAG nanofibers is formulated as an ultra-thin membrane. In a specific embodiment, a composition comprising the sNAG nanofibers is formulated as a suspension or a gel. In preferred embodiments, the suspension or gel is an isotonic suspension or gel. In a specific embodiment, a composition comprising sNAG nanofibers is formulated for injection (e.g., injection into to gingiva).

A composition comprising the sNAG nanofibers may include one or more of pharmaceutically acceptable excipients. Suitable excipients may include water, saline, salt solution, dextrose, glycerol, ethanol and the like, or combinations thereof. Suitable excipients also include starch, glucose, lactose, sucrose, gelatin, malt, rice, flour, chalk, silica gel, sodium stearate, glycerol monostearate, oil (including those of petroleum, animal, vegetable or synthetic origin, such as peanut oil, soybean oil, mineral oil, sesame oil and the like), talc, sodium chloride, dried skim milk, propylene, glycol and the like. In addition, a composition comprising the sNAG nanofibers may include one or more of wetting agents, emulsifying agents, pH buffering agents, and other agents. The sNAG nanofiber compositions may also be incorporated in a physiologically acceptable carrier, for example in a physiologically acceptable carrier suitable for injection. The term “pharmaceutically acceptable” means approved by a regulatory agency of the Federal or a state government or listed in the U.S. Pharmacopeia or other generally recognized pharmacopeia for use in animals, and more particularly in humans. Examples of suitable pharmaceutical carriers are described in “Remington's Pharmaceutical Sciences” by E. W. Martin.

The final amount of the sNAG nanofibers in a composition may vary. For example, the amount of the sNAG nanofibers in a composition (e.g., prepared for administration to a patient) may be greater than or equal to about 50%, about 60%, about 70%, about 75%, about 80%, about 85%, about 90%, about 95%, about 98%, or about 99% weight by volume. In one embodiment, the amount of the sNAG nanofibers in a composition is about 95%, about 98%, about 99, or about 100%. Also, the amount of the sNAG nanofibers in a composition (e.g., prepared for administration to a patient) may be about 50%-100%, about 60%-100%, about 70%-100%, about 75%-100%, about 80%-100%, about 90%-100%, about 95%-100%, about 70%-95%, about 75%-95%, about 80%-95%, about 90%-95%, about 70%-90%, about 75%-90%, or about 80%-90% weight/volume. A composition may comprise more than 30%, 40%, 50%, 60%, 70%, 75%, 80%, 90%, 95% or 99% solution of the sNAG nanofibers.

In some embodiments, the composition is a suspension or a gel comprising approximately 5 to 35 mg of sNAG nanofibers per mL of isotonic solution (e.g., saline or PBS) or water. In some embodiments, in the composition is a suspension or a gel comprising approximately 5 to 30 mg of sNAG nanofibers per mL of isotonic solution (e.g., saline or PBS) or water. In some embodiments, the composition is a suspension or a gel comprising approximately 20 mg of sNAG nanofibers per mL of isotonic solution (e.g., saline or PBS) or water. In other embodiments, the concentration of sNAG nanofibers in the suspension or gel is about 5-35 mg/mL. In other embodiments, the concentration of sNAG nanofibers in the suspension or gel is about 5-30 mg/mL. In other embodiments, the concentration of sNAG nanofibers in the suspension or gel is about 20 mg/mL.

In some embodiments, the composition is a suspension or a gel and the viscosity of the suspension or gel is about 8-12 cP. In certain embodiments, the viscosity of the suspension or gel is about 10 cP. In some embodiments, the viscosity of the suspension or gel is determined by preparing a solution of the suspension or gel as described in Terbojevich et al., “Solution Study of the Chitin-Lithium Chloride-N,N-Dimethylacetamide System,” Carbohydrate Research; 1988; 180:73-86, and taking viscosity measurements of the solution with a rheometer.

In some embodiments, the composition is in a syringe. In a particular embodiment, the composition is in a pre-filled syringe. In other embodiments, the syringe contains 10-100 μL of the sNAG nanofiber composition. In some embodiments, the syringe contains 40-50 μL of the sNAG nanofiber composition. In other embodiments, the syringe contains 40-60 μL of the sNAG nanofiber composition. In some embodiments, the syringe contains 30-50 μL of the sNAG nanofiber composition. In other embodiments, the syringe contains 30-60 μL of the sNAG nanofiber composition. In certain embodiments, the syringe contains 30-40 μL of the sNAG nanofiber composition.

In certain aspects, the sNAG nanofiber is the only active ingredient in a composition. In some embodiments, a sNAG nanofiber composition described herein comprises one, two, or more of the ingredients recited in Section 5.7, infra.

In other embodiments, a composition comprises one or more additional active ingredients, e.g., to promote an anti-bacterial effect, an anti-viral effect, or both. In some embodiments, the one or more additional active ingredients promotes an anti-bacterial effect. In some embodiments, the one or more additional active ingredients promotes an anti-viral effect. In other embodiments, the additional active ingredient is an agent that is a pain relief agent, an antibiotic or an anesthetic.

In certain embodiments, a sNAG nanofiber composition described herein comprises a growth factor to promote regeneration of gingiva tissue (e.g., epithelial or connective tissue). In some embodiments, a sNAG nanofiber composition described herein comprises one, two or more of the following: platelet derived growth factor (PDGF), fibroblast growth factor (FGF) (e.g., acidic or basic FGF, or both), bone morphogenetic protein(s) (BMPs) (e.g., BMP-2), insulin-like growth factor I, insulin-like growth factor II, transforming growth factor β (TGF-β), periodontal ligament derived growth factor, RNAi, or gene therapy. In certain embodiments, a sNAG nanofiber composition described herein comprises one or more of the following: cells (e.g., fibroblasts or epithelial cells, or both), or human fibroblast derived dermal substitute.

A sNAG nanofiber composition may contain collagen, although in certain aspects a sNAG nanofiber composition does not contain collagen.

In certain embodiments, a sNAG nanofiber composition does not comprise any additional therapy. In certain embodiments, a sNAG nanofiber composition does not comprise any additional anti-bacterial agent or anti-viral agent. In some embodiments, a sNAG nanofiber composition does not comprise either an anti-bacterial agent or anti-viral agent. In some embodiments, a sNAG nanofiber composition does not comprise an antibiotic. In yet other embodiments, a sNAG nanofiber composition may comprise an additional therapy (e.g., an antibiotic).

In certain embodiments, a sNAG nanofiber described herein does not comprise a growth factor to promote regeneration of gingiva tissue (e.g., epithelial or connective tissue). In some embodiments, a sNAG nanofiber composition described herein does not comprise any of the following: platelet derived growth factor (PDGF), fibroblast growth factor (FGF) (e.g., acidic or basic FGF, or both), bone morphogenetic protein(s) (BMPs) (e.g., BMP-2), insulin-like growth factor I, insulin-like growth factor II, transforming growth factor β (TGF-β), periodontal ligament derived growth factor, RNAi, or gene therapy. In certain embodiments, a sNAG nanofiber composition described herein does not comprise cells (e.g., fibroblasts or epithelial cells) or human fibroblast derived dermal substitute. In some embodiments, a sNAG nanofiber composition described herein does not comprise cells (e.g., fibroblasts or epithelial cells) or growth factor to promote tissue regeneration.

In other aspects, a sNAG nanofiber composition does not comprise a significant amount of protein material. In specific embodiments, the protein content of a sNAG nanofiber composition is no greater than 0.1%, 0.5% or 1% by weight. In other embodiments, the protein content of the composition is undetectable by Coomassie staining.

In other embodiments, the sNAG nanofiber composition is sterile. In some embodiments, the sNAG nanofiber composition contains no endotoxins. In certain embodiments, the sNAG nanofiber composition is sterile, as measured by methods described in USP <61> (see, e.g., United States Pharmacopeia 41-National Formulary 36). In other embodiments, a packaged sNAG nanofiber composition is terminally sterilized post packaging (see, e.g., “Sterilization of health care products—Radiation,” ISO 11137-2012.).

### 5.4 Use of sNAG Nanofiber Compositions for the Treatment of Gingival Recession

The sNAG nanofiber compositions described herein may be used in accordance with the methods described herein or in the kits described herein. In one aspect, provided herein are methods of treating gingival recession using sNAG nanofibers or a composition thereof. In one embodiment, provided herein are methods for treating gingival recession, the methods comprising administering to the gingiva of a subject (e.g., a human) in need thereof a composition comprising shortened sNAG nanofibers. In certain embodiments, more than 50% of the sNAG nanofibers are between about 1 to 15 μm in length and the sNAG nanofibers comprise glucosamine monosaccharides, and wherein at least 70% of the monosaccharides are N-acetylglucosamine monosaccharides. See, e.g., Section 5.1, supra, and Section 6.3, infra, for further description of the sNAG nanofibers that may be used in the methods. In certain embodiments, the composition administered to the gingiva of the subject is formulated as a suspension or gel. See, e.g., Section 5.3, supra, for a description of such formulations as well as other ways that the composition administered to the gingiva of the subject may be formulated. In certain embodiments, the composition administered to the gingiva of the subject comprises the sNAG nanofibers as the sole active ingredient. In other embodiments, the composition administered to the gingiva of the subject comprises one or more active ingredients, such as described in Section 5.3, supra, and Section 5.7, infra. In a specific embodiment, the composition administered to the gingiva of the subject comprises sNAG nanofibers and a pain reliever or anesthetic.

In another aspect, provided herein are methods of treating a subject displaying a loss of gingiva tissue (e.g., a loss of connective or epithelial tissue, or both), a pocket(s) in the gingiva, or a gingival cleft(s), or a combination of any of the foregoing, using sNAG nanofibers or a composition thereof. In one embodiment, provided herein are methods for treating a loss of gingiva tissue (e.g., a loss of connective or epithelial tissue, or both), a pocket(s) in the gingiva, or a gingival cleft(s), or a combination of any of the foregoing, the methods comprising administering to the gingiva of a subject in need thereof a composition comprising sNAG nanofibers. In certain embodiments, more than 50% of the sNAG nanofibers are between about 1 to 15 μm in length and the sNAG nanofibers comprise glucosamine monosaccharides, and wherein at least 70% of the monosaccharides are N-acetylglucosamine monosaccharides. See, e.g., Section 5.1, supra, and Section 6.3, infra, for further description of the sNAG nanofibers that may be used in the methods. In certain embodiments, the composition administered to the gingiva of the subject is formulated as a suspension or gel. See, e.g., Section 5.3, supra, for a description of such formulations as well as other ways that the composition administered to the gingiva of the subject may be formulated. In certain embodiments, the composition administered to the gingiva of the subject comprises the sNAG nanofibers as the sole active ingredient. In other embodiments, the composition administered to the gingiva of the subject comprises one or more active ingredients, such as described in Section 5.3, supra, and Section 5.7, infra. In a specific embodiment, the composition administered to the gingiva of the subject comprises sNAG nanofibers and a pain reliever or anesthetic.

In a specific embodiment, a composition described herein comprising sNAG nanofibers is injected into the gingiva of the subject. In another specific embodiment, a composition described herein comprising sNAG nanofibers is administered to the coronal gingiva of the subject. In another specific embodiment, described herein comprising sNAG nanofibers is injected into the coronal gingiva of the subject. In a preferred embodiment, an effective amount of described herein comprising sNAG nanofibers is administered to the gingiva (e.g., the coronal gingiva) of the subject. In certain embodiments, the effective amount is able to increase the cement-enamel junction measurement by 1, 2, 3, 4, 5 or 6 mm at the mesial, mid or distal section of the coronal portion of the gingiva. In some embodiments, the effective amount may result in increasing the cement-enamel junction measurement by 1, 2, 3, 4, 5 or 6 mm at two or three of the following: the mesial, mid or distal section of the coronal portion of the gingiva. In certain embodiments, the effective amount may achieve one or more of the following: (1) decrease the reduction in the thinning of the gingiva; (2) prevent a further reduction of the cement-enamel junction measurement at two or three of the following: the mesial, mid or distal section of the coronal portion of the gingiva; or (3) maintain the cement-enamel junction measurement at two or three of the following: the mesial, mid or distal section of the coronal portion of the gingiva. See, e.g., Section 5.6, infra, for amount of sNAGs that may be administered to the gingiva (e.g., the coronal gingiva) to treat gingival recession. In some embodiments, the sNAG nanofibers act as a scaffold for tissue regeneration. In other embodiments, the sNAG nanofibers do not act as a scaffold for tissue regeneration.

In certain embodiments, the gingival recession, loss of gingiva tissue, a pocket(s) in the gingiva, or gingival cleft(s) treated in accordance with the methods described herein affects molars. In some embodiments, the gingival recession, loss of gingiva tissue, a pocket(s) in the gingiva, or gingival cleft(s) treated in accordance with the methods described herein affects incisors (e.g., mandibular incisors). In certain embodiments, the gingival recession, loss of gingiva tissue, a pocket(s) in the gingiva, or gingival cleft(s) treated in accordance with the methods described herein affects canines. In some embodiments, the gingival recession, loss of gingiva tissue, a pocket(s) in the gingiva, or gingival cleft(s) treated in accordance with the methods described herein affects the first and second of maxialla. In certain embodiments, the gingival recession, loss of gingiva tissue, a pocket(s) in the gingiva, or gingival cleft(s) treated in accordance with the methods described herein affects pre-molars. In some embodiments, the gingival recession, loss of gingiva tissue, a pocket(s) in the gingiva, or gingival cleft(s) treated in accordance with the methods described herein is localized. In other embodiments, the gingival recession, loss of gingiva tissue, a pocket(s) in the gingiva, or gingival cleft(s) treated in accordance with the methods described herein is generalized.

In certain embodiments, prior to treatment in accordance with the methods described herein the gingival recession as measured by cement-enamel junction is −1 mm at the mesial, mid or distal location of the coronal gingiva. In some embodiments, prior to treatment in accordance with the methods described herein the gingival recession as measured by cement-enamel junction is −1 mm at the mesial, mid and distal location of the coronal gingiva. In certain embodiments, prior to treatment in accordance with the methods described herein the gingival recession as measured by cement-enamel junction is −1 mm at two of the three locations: mesial, mid or distal location of the coronal gingiva. A periodontal probe may be used to measure the cement-enamel junction. See, e.g., Example 6.2, infra, for measurement with a periodontal probe.

In certain embodiments, prior to treatment in accordance with the methods described herein the gingival recession as measured by cement-enamel junction is −2 mm at the mesial, mid or distal location of the coronal gingiva. In some embodiments, prior to treatment in accordance with the methods described herein the gingival recession as measured by cement-enamel junction is −2 mm at the mesial, mid and distal location of the coronal gingiva. In certain embodiments, prior to treatment in accordance with the methods described herein the gingival recession as measured by cement-enamel junction is −2 mm at two of the three locations: mesial, mid or distal location of the coronal gingiva. A periodontal probe may be used to measure the cement-enamel junction.

In certain embodiments, prior to treatment in accordance with the methods described herein the gingival recession as measured by cement-enamel junction is −3 mm at the mesial, mid or distal location of the coronal gingiva. In some embodiments, prior to treatment in accordance with the methods described herein the gingival recession as measured by cement-enamel junction is −3 mm at the mesial, mid and distal location of the coronal gingiva. In certain embodiments, prior to treatment in accordance with the methods described herein the gingival recession as measured by cement-enamel junction is −3 mm at two of the three locations: mesial, mid or distal location of the coronal gingiva. A periodontal probe may be used to measure the cement-enamel junction.

In certain embodiments, prior to treatment in accordance with the methods described herein the gingival recession as measured by cement-enamel junction is −4 mm at the mesial, mid or distal location of the coronal gingiva. In some embodiments, prior to treatment in accordance with the methods described herein the gingival recession as measured by cement-enamel junction is −4 mm at the mesial, mid and distal location of the coronal gingiva. In certain embodiments, prior to treatment in accordance with the methods described herein the gingival recession as measured by cement-enamel junction is −4 mm at two of the three locations: mesial, mid or distal location of the coronal gingiva. A periodontal probe may be used to measure the cement-enamel junction.

In certain embodiments, prior to treatment in accordance with the methods described herein the gingival recession as measured by cement-enamel junction is −5 mm at the mesial, mid or distal location of the coronal gingiva. In some embodiments, prior to treatment in accordance with the methods described herein the gingival recession as measured by cement-enamel junction is −5 mm at the mesial, mid and distal location of the coronal gingiva. In certain embodiments, prior to treatment in accordance with the methods described herein the gingival recession as measured by cement-enamel junction is −5 mm at two of the three locations: mesial, mid or distal location of the coronal gingiva. A periodontal probe may be used to measure the cement-enamel junction.

In certain embodiments, prior to treatment in accordance with the methods described herein the gingival recession as measured by cement-enamel junction is −6 mm at the mesial, mid or distal location of the coronal gingiva. In some embodiments, prior to treatment in accordance with the methods described herein the gingival recession as measured by cement-enamel junction is −6 mm at the mesial, mid and distal location of the coronal gingiva. In certain embodiments, prior to treatment in accordance with the methods described herein the gingival recession as measured by cement-enamel junction is −6 mm at two of the three locations: mesial, mid or distal location of the coronal gingiva. A periodontal probe may be used to measure the cement-enamel junction.

In certain embodiments, prior to treatment in accordance with the methods described herein the gingival recession as measured by cement-enamel junction is between −1 and −6 mm, −2 and −6 mm, or −2 and −5 mm at the mesial, mid or distal location of the coronal gingiva. In some embodiments, prior to treatment in accordance with the methods described herein the gingival recession as measured by cement-enamel junction is between −1 and −6 mm, −2 and −6 mm, or −2 and −5 mm at the mesial, mid and distal location of the coronal gingiva. In certain embodiments, prior to treatment in accordance with the methods described herein the gingival recession as measured by cement-enamel junction is between −1 and −6 mm, −2 and −6 mm, or −2 and −5 mm at two of the three locations: mesial, mid or distal location of the coronal gingiva. A periodontal probe may be used to measure the cement-enamel junction.

In certain embodiments, the gingival recession treated in accordance with the methods described herein is classified as class I under Miller's classification system. See Miller, 1985, International Journal of Periodontics and Restorative Dentistry 5(2): 8-13 for description of Miller's classification system. In some embodiments, the gingival recession treated in accordance with the methods described herein is classified as class II under Miller's classification system. In certain embodiments, the gingival recession treated in accordance with the methods described herein is classified as class III under Miller's classification system. In some embodiments, the gingival recession treated in accordance with the methods described herein is classified as class IV under Miller's classification system.

In some embodiments, a composition described herein for use in accordance with the methods described herein is administered by a dentist to a patient. In particular embodiments, the composition is administered by the dentist following a check-up or cleaning.

In particular embodiments, treatment of the gingiva in accordance with the methods described herein achieves one, two or more of the following: (1) a decrease in the reduction in the thinning of the gingiva; (2) prevention of a further reduction of the cement-enamel junction measurement at two or three of the following: the mesial, mid or distal section of the coronal portion of the gingiva; (3) maintenance of the cement-enamel junction measurement at two or three of the following: the mesial, mid or distal section of the coronal portion of the gingiva; (4) reduction of the likelihood of the need for gingival grafting, guided tissue regeneration, or orthodontic therapy; (5) a reduction in pocket formation in the gingiva; (6) a reduction in gingival clefts; (7) a decrease in the number of symptoms of gingival recession; (8) an increase in the thickness of the epithelium of the injected gingiva; or (9) an increase in the thickness of the connective tissue of the injected gingiva; or (10) a reduction in bacterial plaque.

In certain embodiments, a composition described herein is administered to the gingiva of a subject treat gingival recession, loss of gingiva tissue, a pocket(s) in the gingiva, or gingival cleft(s) alone or in combination with another therapy. See, e.g., Section 5.7 for therapies that may be administered to a subject in conjunction with a composition described herein. In a specific embodiment, a composition described herein is administered alone to the gingiva of a subject treat gingival recession, loss of gingiva tissue, a pocket(s) in the gingiva, or gingival cleft(s). In certain embodiments, a composition described herein is administered to the gingiva of a subject to treat gingival recession, loss of gingiva tissue, a pocket(s) in the gingiva, or gingival cleft(s) in conjunction with the administration of a pain reliever or anesthetic.

### 5.5 Patient Populations

In one embodiment, sNAG nanofibers or a sNAG nanofiber composition described herein may be administered to a patient who has been diagnosed with gingival recession. In another embodiment, sNAG nanofibers or a sNAG nanofiber composition described herein may be administered to a patient who displays one or more symptoms of gingival recession. For example, a composition described herein may be administered to a patient that displays a thinning of the gums, a loss of gingiva tissue, a pocket(s) in the gingiva, or a gingival cleft(s).

In some embodiments, a subject to be administered sNAG nanofibers or a sNAG nanofiber composition described herein is an animal. In certain embodiments, the animal is a canine. In certain embodiments, the animal is a feline. In certain embodiments, the animal is a horse. In certain embodiments, the animal is a cow. In certain embodiments, the animal is a mammal, e.g., a horse, swine, mouse, or primate, preferably a human. In some embodiments, the animal is a pet or a farm animal.

In certain embodiments, a subject to be administered sNAG nanofibers or a sNAG nanofiber composition described herein is a human adult. In certain embodiments, a subject to be administered sNAG nanofibers or a sNAG nanofiber composition described herein is a human adult more than 50 years old. In certain embodiments, a subject to be administered sNAG nanofibers or a sNAG nanofiber composition described herein is an elderly human subject. In certain embodiments, a subject to be administered sNAG nanofibers or a sNAG nanofiber composition described herein is a human child.

In certain embodiments, a subject to be administered sNAG nanofibers or a sNAG nanofiber composition described herein has a cement-enamel junction measurement of −1 mm at the mesial, mid or distal location of the coronal gingiva. In some embodiments, a subject to be administered sNAG nanofibers or a sNAG nanofiber composition described herein has a cement-enamel junction measurement of −1 mm at the mesial, mid and distal location of the coronal gingiva. In certain embodiments, a subject to be administered sNAG nanofibers or a sNAG nanofiber composition described herein has a cement-enamel junction measurement of −1 mm at two of the three locations: mesial, mid or distal location of the coronal gingiva.

In certain embodiments, a subject to be administered sNAG nanofibers or a sNAG nanofiber composition described herein has a cement-enamel junction measurement of −2 mm at the mesial, mid or distal location of the coronal gingiva. In some embodiments, a subject to be administered sNAG nanofibers or a sNAG nanofiber composition described herein has a cement-enamel junction measurement of −2 mm at the mesial, mid and distal location of the coronal gingiva. In certain embodiments, a subject to be administered sNAG nanofibers or a sNAG nanofiber composition described herein has a cement-enamel junction measurement of −2 mm at two of the three locations: mesial, mid or distal location of the coronal gingiva.

In certain embodiments, a subject to be administered sNAG nanofibers or a sNAG nanofiber composition described herein has a cement-enamel junction measurement of −3 mm at the mesial, mid or distal location of the coronal gingiva. In some embodiments, a subject to be administered sNAG nanofibers or a sNAG nanofiber composition described herein has a cement-enamel junction measurement of −3 mm at the mesial, mid and distal location of the coronal gingiva. In certain embodiments, a subject to be administered sNAG nanofibers or a sNAG nanofiber composition described herein has a cement-enamel junction measurement of −3 mm at two of the three locations: mesial, mid or distal location of the coronal gingiva.

In certain embodiments, a subject to be administered sNAG nanofibers or a sNAG nanofiber composition described herein has a cement-enamel junction measurement of −4 mm at the mesial, mid or distal location of the coronal gingiva. In some embodiments, a subject to be administered sNAG nanofibers or a sNAG nanofiber composition described herein has a cement-enamel junction measurement of −4 mm at the mesial, mid and distal location of the coronal gingiva. In certain embodiments, a subject to be administered sNAG nanofibers or a sNAG nanofiber composition described herein has a cement-enamel junction measurement of −4 mm at two of the three locations: mesial, mid or distal location of the coronal gingiva.

In certain embodiments, a subject to be administered sNAG nanofibers or a sNAG nanofiber composition described herein has a cement-enamel junction measurement of −5 mm at the mesial, mid or distal location of the coronal gingiva. In some embodiments, a subject to be administered sNAG nanofibers or a sNAG nanofiber composition described herein has a cement-enamel junction measurement of −5 mm at the mesial, mid and distal location of the coronal gingiva. In certain embodiments, a subject to be administered sNAG nanofibers or a sNAG nanofiber composition described herein has a cement-enamel junction measurement of −5 mm at two of the three locations: mesial, mid or distal location of the coronal gingiva.

In certain embodiments, a subject to be administered sNAG nanofibers or a sNAG nanofiber composition described herein has a cement-enamel junction measurement of −6 mm at the mesial, mid or distal location of the coronal gingiva. In some embodiments, a subject to be administered sNAG nanofibers or a sNAG nanofiber composition described herein has a cement-enamel junction measurement of −6 mm at the mesial, mid and distal location of the coronal gingiva. In certain embodiments, a subject to be administered sNAG nanofibers or a sNAG nanofiber composition described herein has a cement-enamel junction measurement of −6 mm at two of the three locations: mesial, mid or distal location of the coronal gingiva.

In certain embodiments, a subject to be administered sNAG nanofibers or a sNAG nanofiber composition described herein has a cement-enamel junction measurement of between −1 and −6 mm, −2 and −6 mm, or −2 and −5 mm at the mesial, mid or distal location of the coronal gingiva. In some embodiments, a subject to be administered sNAG nanofibers or a sNAG nanofiber composition described herein has a cement-enamel junction measurement of between −1 and −6 mm, −2 and −6 mm, or −2 and −5 mm at the mesial, mid and distal location of the coronal gingiva. In certain embodiments, a subject to be administered sNAG nanofibers or a sNAG nanofiber composition described herein has a cement-enamel junction measurement of between −1 and −6 mm, −2 and −6 mm, or −2 and −5 mm at two of the three locations: mesial, mid or distal location of the coronal gingiva.

In certain embodiments, a subject to be administered sNAG nanofibers or a sNAG nanofiber composition described herein has gingival recession that is classified as class I under Miller's classification system. See Miller, 1985, International Journal of Periodontics and Restorative Dentistry 5(2): 8-13 for description of Miller's classification system. In some embodiments, a subject to be administered sNAG nanofibers or a sNAG nanofiber composition described herein has gingival recession that is classified as class II under Miller's classification system. In certain embodiments, a subject to be administered sNAG nanofibers or a sNAG nanofiber composition described herein has gingival recession that is classified as class III under Miller's classification system. In some embodiments, a subject to be administered sNAG nanofibers or a sNAG nanofiber composition described herein has gingival recession that is classified as class IV under Miller's classification system. In certain embodiments, a subject to be administered sNAG nanofibers or a sNAG nanofiber composition described herein has a biotype I gingival thickness.

In some embodiments, sNAG nanofibers or a sNAG nanofiber composition described herein may be administered to a patient who is a candidate for regeneration of the gingiva. In certain embodiments, sNAG nanofibers or a sNAG nanofiber composition described herein may be administered to a patient diagnosed with gingival recession or displays one or more symptoms of gingival recession, wherein the gingival recession may have been caused, at least in part, by alveolar bone dehiscence, high muscle attachment, frenum pull and ocular trauma, prosthetic treatment, smoking, poor hygiene, gingival inflammation, or clinical attachment loss. In some embodiments, sNAG nanofibers or a sNAG nanofiber composition described herein may be administered to a patient diagnosed with gingival recession or displays one or more symptoms of gingival recession, wherein the gingival recession may have been caused, at least in part, by bacterial plaque. In some embodiments, sNAG nanofibers or a sNAG nanofiber composition described herein may be administered to a patient diagnosed with gingival recession or displays one or more symptoms of gingival recession, wherein the gingival recession is not caused by bacterial plaque.

In certain embodiments, sNAG nanofibers or a sNAG nanofiber composition described herein may be administered to a patient has not been not diagnosed with a bacterial, fungal, parasitic or viral infection. In particular, sNAG nanofibers or a sNAG nanofiber composition described herein may be administered to a patient has not been not diagnosed with a bacterial, fungal, parasitic or viral infection affecting the gingiva. In some embodiments, sNAG nanofibers or a sNAG nanofiber composition described herein may be administered to a patient does not display one or more symptoms of a bacterial, fungal, parasitic or viral infection. In particular, sNAG nanofibers or a sNAG nanofiber composition described herein may be administered to a patient does not display one or more symptoms of a bacterial, fungal, parasitic or viral infection affecting the gingiva.

In certain embodiments, the gingiva being treated is not wounded.

### 5.6 Modes of Administration of sNAG Nanofiber Compositions

In some embodiments, sNAG nanofibers or a composition comprising the sNAG nanofibers is administered to the site of a gingival recession, pocket formation, gingival cleft or gum thinning in a patient. In certain embodiments, sNAG nanofibers or a composition comprising the sNAG nanofibers is administered within 500 microns, 750 microns, 1 mm, or 2 mm of the site of gingival recession, pocket formation, gingival cleft or gum thinning in a patient. In some embodiments, the site of a gingival recession, pocket formation, gingival cleft or gum thinning is by molars. In certain embodiments, the site of a gingival recession, pocket formation, gingival cleft or gum thinning is by incisors (e.g., mandibular incisors). In some embodiments, the site of a gingival recession, pocket formation, gingival cleft or gum thinning is by canines. In some embodiments, the site of a gingival recession, pocket formation, gingival cleft or gum thinning is by the first and second of maxialla. In certain embodiments, the site of a gingival recession, pocket formation, gingival cleft or gum thinning is by pre-molars.

In specific embodiment, sNAG nanofibers or a composition comprising sNAG nanofibers is administered by injection to the gingiva. In a particular embodiment, sNAG nanofibers or a composition comprising sNAG nanofibers is administered by injection to the coronal gingiva.

In another embodiment, sNAG nanofibers or a sNAG nanofiber composition described herein may be applied topically to a surface of the gingiva of a patient. For example, such composition may be applied topically to coronal gingiva.

The above-listed methods for administration may include administration of the sNAG nanofiber or composition thereof in the form of a cream, an ointment, suspension, a gel, a liquid solution, a membrane, a film, a spray, or any other formulation described herein or known in the art. In a specific embodiment, the composition for administration is formulated as a suspension or gel.

Contemplated treatment regimens include a single dose or a single application of sNAG nanofibers or a sNAG nanofiber composition (e.g., of a suspension or gel), or a regiment of multiple doses or multiple applications of a sNAG nanofiber composition. A dose or an application may be administered daily, weekly or monthly. For example, a dose of a sNAG nanofiber composition may be administered every 48 hours, every 72 hours, once a week, 2 times a week, 3 times a week, every other day, once in 2 weeks, once in 3 weeks, once in 4 weeks, or once a month. In certain embodiments, a dose of sNAG nanofiber composition is administered three times during dental visits. Those three times may be 2 weeks, 1 month, 2 months, 3 months, 4 months, 6 months or more apart from each other. See, e.g., Sections 6.1 and 6.2 regarding dosing of Talymed to subjects. In a specific embodiment, a sNAG nanofiber composition may be administered using a dosing regimen described in Section 6.1 or 6.2, infra.

sNAG nanofibers or a sNAG nanofiber composition may be administered for a duration equal to or greater than 1 week, 2 weeks, 3 weeks, 1 month, 2 months, 3 months, 4 months, 5 months, 6 months, 9 months, 1 year, 1.5 years, 2 years, 2.5 years, 3 years, 4 years, 5 years, 7 years, 10 years or more. In one such embodiment, sNAG nanofibers or a sNAG nanofiber composition does not cause any side effects or causes only mild side effects during the duration of the treatment. In another embodiment, sNAG nanofibers or a sNAG nanofiber composition does not cause irritation (e.g., moderate or severe irritation) or allergy (e.g., moderate or severe allergy).

Concentration of the sNAG nanofiber in a composition may vary. In general, an effective amount of the sNAG nanofiber is used. An effective amount may be an amount sufficient to achieve one or more of the effects described herein. For example, a composition may comprise about 5 to 30 mg of the sNAG nanofibers per mL of an isotonic solution in a form suitable for administration to a patient. In certain embodiments, a composition described herein comprises about 5 to 25 mg of the sNAG nanofibers per mL of an isotonic solution, 5 to 20 mg of the sNAG nanofibers per mL of an isotonic solution, about 5 to 15 mg of the sNAG nanofibers per mL of an isotonic solution or about 5 to 10 mg of the sNAG nanofibers per mL of an isotonic solution. In other embodiments, the concentration of sNAG nanofibers in the composition is about 5-35 mg/mL. In other embodiments, the concentration of sNAG nanofibers in the composition is about 5-30 mg/mL. In other embodiments, the concentration of sNAG nanofibers in the composition is about 5-20 mg/mL. In other embodiments, the concentration of sNAG nanofibers in the suspension or gel is about 20 mg/mL. In particular embodiments, a dose of 30 to 40 microliters of a composition described herein is administered to the gingiva of a subject. In a specific embodiment, a dose of 30 to 40 microliters of a composition described herein is administered to the gingiva of a subject, wherein the concentration of sNAG nanofibers in the composition is 5 to 30 mg/mL. In a particular embodiment, the composition is an isotonic suspension or an isotonic gel. In certain embodiments, 0.1 to 2 mg, 0.15 to 0.2 mg, 0.1 to 0.2 mg, 0.9 to 1.2 mg, 0.15 to 1.2 mg, 0.10 to 1.5 mg, or 0.10 to 2 mg of sNAG nanofibers are administered to a subject as a dose per gingival recession, pocket or gingival cleft.

### 5.7 Combination Therapy

The sNAG nanofibers or sNAG nanofiber compositions may be administered in conjunction with other therapies such as, e.g., substances that boost the immune system, antibacterial agents (e.g., an antibiotic), defensin peptides, defensin-like peptides, zinc, antivirals, pain relief therapy (e.g., an analgesic), fever relief therapy. Alternatively, the sNAG nanofibers or a composition thereof is not administered in conjunction with any of the preceding therapies.

In some embodiments, sNAG nanofibers or a sNAG nanofiber composition described herein is administered in conjunction with an anti-bacterial agent, for example an antibiotic. In other embodiments, a composition described herein is not administered in conjunction with an anti-bacterial agent, for example an antibiotic. In some embodiments, sNAG nanofibers or a sNAG nanofiber composition described herein is administered in conjunction with an anti-viral agent or anti-fungal agent. In other embodiments, sNAG nanofibers or a sNAG nanofiber composition described herein is not administered in conjunction with anti-viral agent or anti-fungal agent.

In some embodiments, sNAG nanofibers or a sNAG nanofiber composition described herein is administered in conjunction with one, two or more of the following: collagen, platelet derived growth factor (PDGF), fibroblast growth factor, bone morphogenetic protein (e.g., bone morphogenetic protein-2), insulin-like growth factor I or II, transforming growth factor (TGF)-beta, periodontal ligament derived growth factor, gene therapy, implantation of cells (e.g., fibroblasts), RNAi, human fibroblast derived dermal substitute, bilayer cell therapy, human cultured gingival epithelial sheets, or metronidazole. In other embodiments, sNAG nanofibers or a sNAG nanofiber composition described herein is not administered in conjunction with one, two or more of the following: collagen, platelet derived growth factor (PDGF), fibroblast growth factor, bone morphogenetic protein (e.g., bone morphogenetic protein-2), insulin-like growth factor I or II, transforming growth factor (TGF)-beta, periodontal ligament derived growth factor, gene therapy, implantation of cells (e.g., fibroblasts), RNAi, human fibroblast derived dermal substitute, bilayer cell therapy, human cultured gingival epithelial sheets, or metronidazole.

In some embodiments, the sNAG nanofibers or sNAG nanofiber compositions described herein are administered before (e.g., 1 minute, 15 minutes, 30 minutes, 1 hour, 2 hours, 3 hours, 6 hours, 12 hours, 24 hours or more before, or any time period in between), simultaneously with, or after (e.g., 1 minute, 15 minutes, 30 minutes, 1 hour, 2 hours, 3 hours, 6 hours, 12 hours, 24 hours or more after, or any time period in between) administration of another therapy. For an example, such sNAG nanofibers or sNAG nanofiber compositions maybe administered before, simultaneously with or after administration of an anti-bacterial agent (e.g., an antibiotic).

### 5.8 Kits

A pharmaceutical pack or kit which comprises any of the above-described sNAG compositions is also contemplated. The pack or kit may comprise one or more containers filled with one or more ingredients comprising the compositions described herein. The composition is preferably contained within a sealed, waterproof, sterile package which facilitates removal of the composition without contamination. Materials from which containers may be made include aluminum foil, plastic, or another conventional material that is easily sterilized. The kit can contain material for a single administration or multiple administrations of the composition, preferably wherein the material for each administration is provided in a separate, waterproof, sterile package.

Additionally, a kit can also contain a syringe(s) for administration of a composition described herein. In some embodiments, a kit contains a syringe pre-filled with a composition described herein.

Optionally associated with such kit or pack can be a notice in the form prescribed by a governmental agency regulating the manufacture, use or sale of pharmaceuticals or biological products, which notice reflects approval by the agency of manufacture, use or sale for human administration. For example, a kit can comprise a notice regarding FDA approval and/or instructions for use.

The kits encompassed herein can be used in the above applications and methods.

### 6. EXAMPLES

### 6.1 Example 1: Effect of Talymed Gel on Gingival Tissue

This example demonstrates that Talymed (T.M.) gel increases the thickness of epithelium and connective tissue of gingiva tissue.

6.1.1 Materials and Methods

Talymed (T.M.) gel is produced and supplied by Marine Polymer Technologies.

Animals: Twenty C57BL/6B mice (13 Wks old) were divided into two groups (n=10): group A was injected using insulin-syringes with 1.5 micro liter (μL) of T.M. gel into the gingiva at the lower central incisors region; group B injected with same amount of PBS only, as a control group. The animals were then left for 6 weeks and given standard rodent chow and water ad libitum. All injections were carried out under general anesthesia obtained using Ketamine/Xylazine combination at standard dosage. All procedures accomplished under surgical microscopes.

Samples Preparations: After six weeks, animals were sacrificed according to the standard protocol, and then samples were immediately fixed with 2% Paraformaldehyde in PBS for 2 hours at room temperature. Following that, samples were decalcified in 10% EDTA-10% Glycerol at pH 7 in PBS for 28 days. Once decalcification completed, blocks of the tissue were prepared for sections in O.C.T. compound and via standard Cryostats a thickness of 5-8 μm were obtained. Before analyses, slides were stained with H&E staining and saved for examinations.

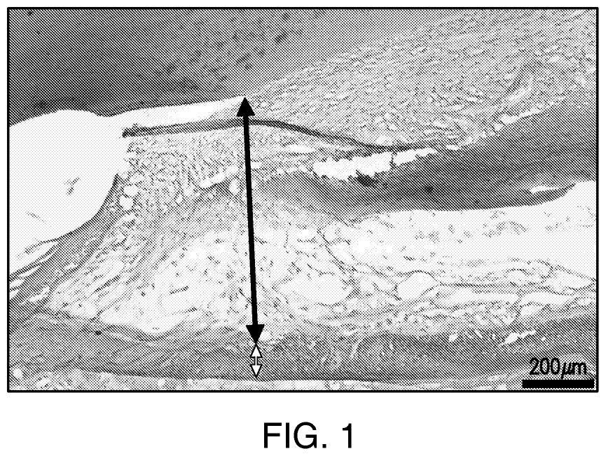

Measurements and Statistical analyses: Three consequent sections from each samples (totaling of 30 data point per group) were obtained at data collection. From each slide, photos were taken under 50× magnification lenses via a microscope-camera. Then, ImagePro Premier Software was used to analyze the data from the images. The pictures on which the measurement collected contained gingival tissue (epithelium and connective tissue), bone crest as well as tooth structure. Then, the thickness of epithelium and connective tissue above the bone crest were evaluated using to the tip of the bone crest as landmark, as shown in FIG. 1. Results considered statistical significant only if the p value>0.05 operating standard T-test.

6.1.2 Results

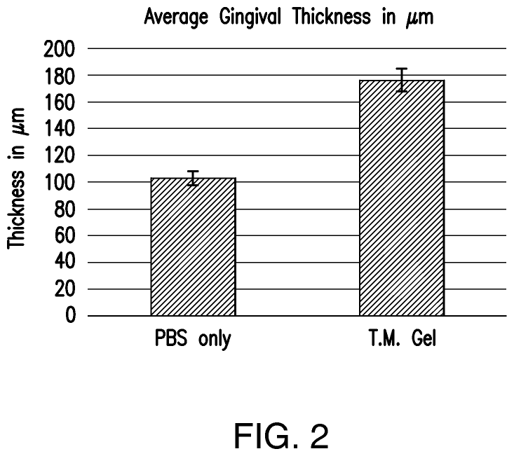

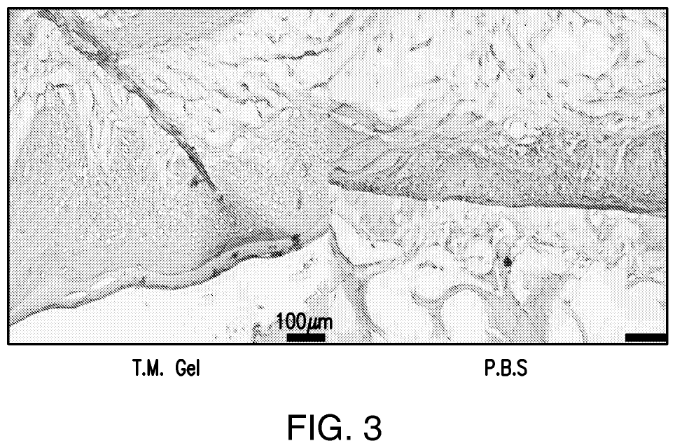

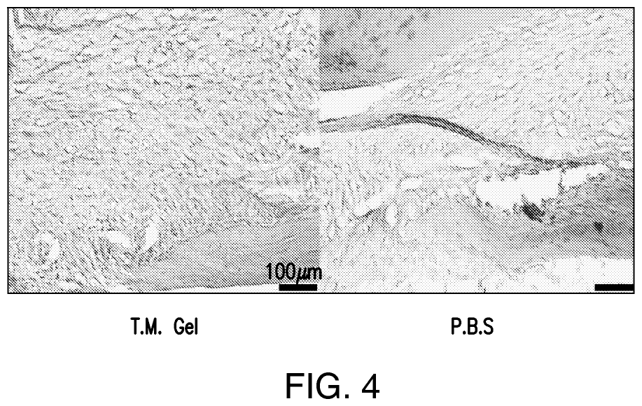

The data show that after 6 weeks Talymed gel was completely resorbed. In addition, Talymed gel injections produced a statistically significant increase in the thickness of the gingival tissue (Ep+CT) 6 weeks after injections. FIG. 2 and FIG. 4 show the increased thickness of the connective tissue after Talymed gel injections. FIG. 3 shows the increased thickness of the epithelium tissue after Talymed gel injections.

6.1.3 Conclusions

In conclusion, the data from the mouse model support that, after six weeks, Talymed gel injection generated a significantly greater gingival thickness complex than control area injected with PBS.

The data demonstrate potential use of Talymed to treat subjects with biotype I gingival thickness who are known to be at greater risk for gingival recession is proposed.

### 6.2 Example 2: Effect of Talymed on Gingival Recession in Human Subjects

This example demonstrates that sNAG nanofibers can reverse gum recession or reduce additional loss of gingiva in human subjects.

6.2.1 Design

Four subjects with gum recession are enrolled in the study.

Exclusion Criteria:
1. Subjects having clinically significant or unstable organic disease, patients having compromised healing potential such as those with diabetes mellitus or connective tissue disorders
2. Subjects with active infectious diseases such as hepatitis, human immunodeficiency virus or tuberculosis
3. Subjects undergoing periodontal treatment (surgical or non-surgical) within 2 month period to enrollment
4. Pregnant or breast feeding women
5. Participation in another clinical trial.

Talymed (T.M.) gel is produced and supplied by Marine Polymer Technologies,

6.2.2 Study Procedure

All probing is recorded by the same examiner. Clinical gum measurements are recorded before the treatment and every other week for eight weeks.

Day 1:

Professional elimination of supra- and subgingival plaque.

Using a periodontal probe measure the gum recession at mesial, mid and distal locations of the coronal gingiva. The Cement Enamel Junction is considered the 0 level, above that level is positive and below negative. Document on the subject trial form the distance in millimeters from the top of the pocket to the bottom of the pocket.

Select a pocket for treatment and document it on the subject trial form. Inject 120 μL of Talyderm Gel at the coronal portion of the gingiva divided into 3 injections of 40 μL each at the mesial, mid and distal section of the coronal portion of the gingiva.

The pocket site is monitored and measured every other week for 8 weeks:

Using a periodontal probe measure the gum recession at mesial, mid and distal locations of the coronal gingiva is measured. The Cement Enamel Junction is considered the 0 level, above that level is positive and below negative. Document on the subject trial form the distance in millimeters from the top of the pocket to the bottom of the pocket.

6.2.3 Results

Measurements: mesial, mid and distal section of the coronal portion of the gingiva.

Patient #1 LD, Female 43 years old, Tooth #20

| | |
|---|---|
| Initial | −2, −2, −2 |
| 2 weeks | −2, −2, −2 |
| 4 weeks | −1, −1, −1 |
| 6 weeks | 0, −1, 1 |

Patient #2 NG Female 54 years old, Tooth #11

| | |
|---|---|
| Initial | −1, −2, −1 |
| 2 weeks | 0, −1, −1 |
| 4 weeks | 0, −1, 0 |
| 6 weeks | 0, −1, 0 |

Patient #3 IM Female 44 years old, Tooth #14

| | |
|---|---|
| Initial | −2, −6, −4 |
| 2 weeks | −2, −4, −3 |
| 4 weeks | −2, −5, −3 |
| 6 weeks | −2, −5, −3 |

Patient #4 WS, Male 43 years old, Tooth #3

| | |
|---|---|
| Initial | −3, −1, −1 |
| 2 weeks | −3, −1, −1 |
| 4 weeks | −1, −2, −1 |
| 6 weeks | −1, −2, −1 |

### 6.3 Example 3: Effect of Irradiation on sNAG Membranes

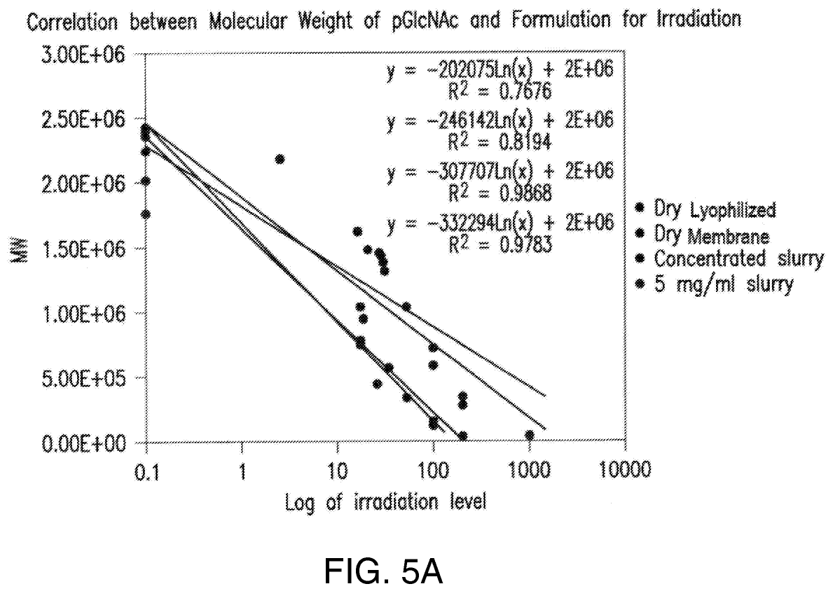

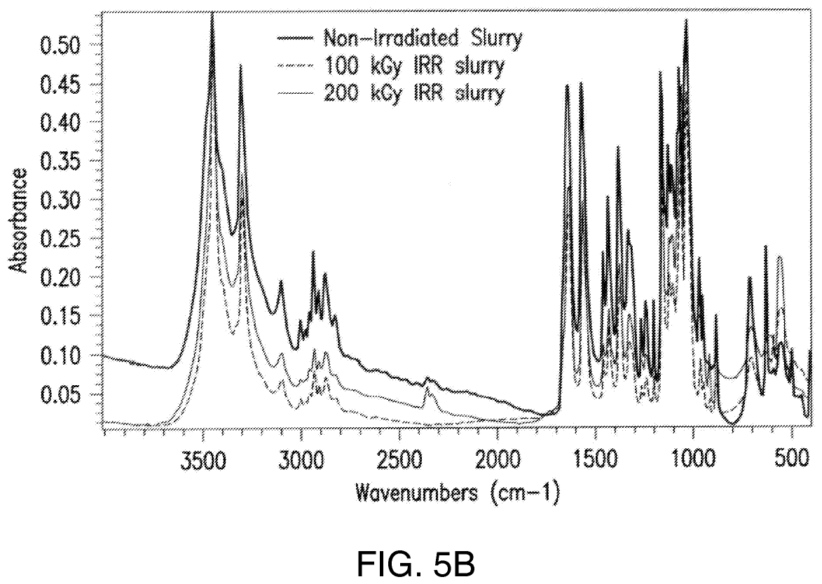

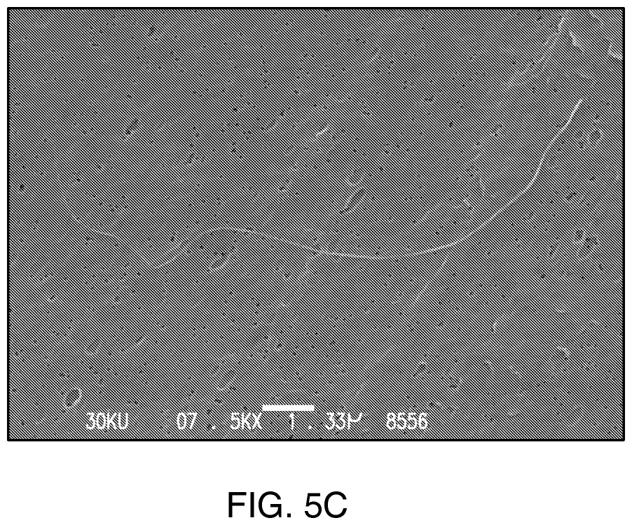

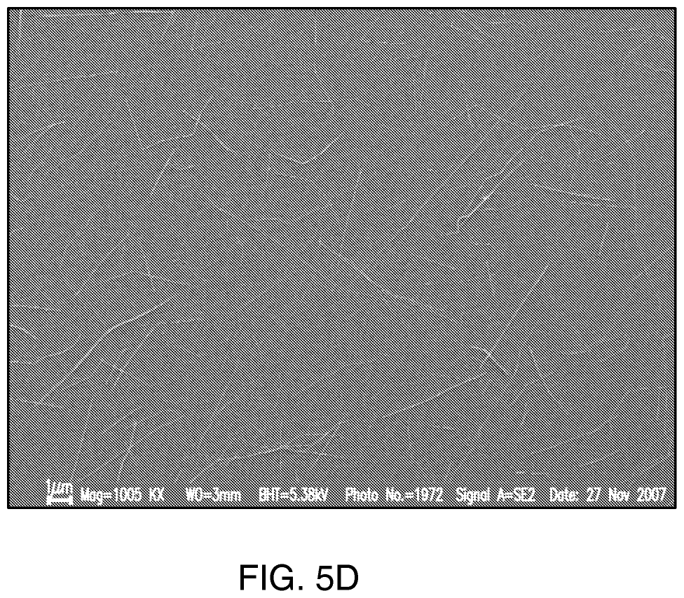

Method of Preparation of sNAG Membrane. The sNAG membrane is derived from microalgal pGlcNAc fibers produced as previously described (see Vournakis et al. U.S. Pat. Nos. 5,623,064; and 5,624,679, the content of each of which is incorporated herein by reference in its entirety). Briefly, microalgae were cultured in unique bioreactor conditions using a defined growth media. Following the harvest of microalgae from high-density cultures, fibers were isolated via a stepwise separation and purification process resulting in batches of pure fibers suspended in water for injections (wfi). Fibers were formulated into patches by concentration and oven drying, and were packaged and sterilized by gamma-irradiation. Fiber dimensions average 20-50 nm×1-2 nm×100 μm. Batches of fibers were individually quality controlled using chemical and physical test parameters, and each batch met strict purity criteria prior to release. Final batches were required to be substantially free of proteins, metal ions, and other components. The fibers were then shortened by irradiation to produce sNAG membranes. Briefly, the starting material contained 60 g of pGlcNAc slurry at a concentration of 1 mg/mL. The concentration of the pGlcNAc slurry was confirmed by filtering 5 mL into a 0.2 um filter. 15 L of pGlcNAc slurry containing 15 g pGlcNAc was filtered until formation of a wet cake. The wake cake was then transferred into a foil pouch, which is a gamma radiation compatible container, and subjected to 200 kGy gamma radiation. Other irradiation conditions were tested for their effects on pGlcNAc compositions, as reflected in FIG. 5A.

Effect of Irradiation on pGlcNAc Membranes. While irradiation reduces the molecular weight of pGlcNAc, irradiation did not disturb the microstructure of the fibers. pGlcNAc was irradiated under different conditions: as a dry, lyophilized material; as a dry membrane; as a concentrated slurry (30:70 weight by volume); and as a dilute slurry (5 mg/ml). A suitable molecular weight reduction (to a molecular weight of 500,000-1,000,000 daltons) was achieved at an irradiation dose of 1,000 kgy for dry polymer, and 200 kgy for wet polymer (FIG. 5A).

The chemical and physical structure of the fibers was maintained throughout irradiation as verified by infrared (IR) spectrum (FIG. 5B), elemental assay, and scanning electron microscopes (SEMs) analysis. Microscopic observation of irradiated fibers showed a decrease in the particle length (FIGS. 5C and 5D). The majority of the fibers are less than about 15 μm in length, with an average length of about 4 um.

### 7. INCORPORATION BY REFERENCE

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for treating gingival recession, comprising injecting into the gingiva of a subject in need thereof a composition comprising shortened fibers of poly-N-acetylglucosamine (“sNAG nanofibers”), wherein more than 50% of the sNAG nanofibers are between about 1 to 15 μm in length, and wherein the sNAG nanofibers comprise glucosamine monosaccharides, and wherein at least 70% of the monosaccharides are N-acetylglucosamine monosaccharides.

2. The method of claim 1, wherein gingival recession affects molars.

3. The method of claim 1, wherein the gingival recession affects incisors.

4. The method of claim 1, wherein the gingival recession affects canines.

5. The method of claim 1, wherein the gingival recession affects pre-molars.

6. The method of any one of claims 1 to 5, wherein the gingival recession is localized.

7. The method of any one of claims 1 to 5, wherein the gingival recession is generalized.

8. The method of any one of claims 1 to 7, wherein the gingival recession prior to injection of the composition as measured by cement-enamel junction is −1 mm at the mesial, mid or distal section of the coronal gingiva.

9. The method of any one of claims 1 to 7, wherein the gingival recession prior to injection of the composition as measured by cement-enamel junction is −2 mm at the mesial, mid or distal section of the coronal gingiva.

10. The method of any one of claims 1 to 7, wherein the gingival recession prior to injection of the composition as measured by cement-enamel junction is −3 mm at the mesial, mid or distal section of the coronal gingiva.

11. The method of any one of claims 1 to 7, wherein the gingival recession prior to injection of the composition as measured by cement-enamel junction is −4 mm at the mesial, mid or distal section of the coronal gingiva.

12. The method of any one of claims 1 to 7, wherein the gingival recession prior to injection of the composition as measured by cement-enamel junction is −5 mm at the mesial, mid or distal section of the coronal gingiva.

13. The method of any one of claims 1 to 7, wherein the gingival recession prior to injection of the composition as measured by cement-enamel junction is −6 mm at the mesial, mid or distal section of the coronal gingiva.

14. The method of any one of claims 1 to 7, wherein the gingival recession prior to injection of the composition as measured by cement-enamel junction is −2 mm to −6 mm at the mesial, mid or distal section of the coronal gingiva.

15. The method of any one of claims 1 to 7, wherein the gingival recession is classified as class I under Miller's classification system.

16. The method of claim 1, wherein the gingival recession is classified as class II under Miller's classification system.

17. The method of claim 1, wherein the gingival recession is classified as class III under Miller's classification system.

18. The method of claim 1, wherein the gingival recession is classified as class IV under Miller's classification system.

19. The method of any one of claims 1 to 18, wherein the sNAG nanofibers more than 50% of the sNAG nanofibers are between about 2 to 10 μm in length.

20. The method of any one of claims 1 to 18, wherein the sNAG nanofibers have an average length of between about 4 to 7 μm in length.

21. The method of any one of claims 1 to 18, wherein 100% of the sNAG nanofibers are between about 1 to 15 μm in length.

22. The method of any one of claims 1 to 21, wherein the length of the sNAG nanofibers is measured by scanning electron microscopy.

23. The method of any one of claims 1 to 22, wherein the sNAG nanofibers were produced by gamma irradiation of poly-N-acetylglucosamine fibers, and wherein the poly-N-acetylglucosamine fibers were irradiated in the form of dried fibers at 500-2,000 kgy, or the poly-β-N-acetylglucosamine fibers were irradiated in the form of wet fibers at 100-500 kgy.

24. The method of any one of claims 1 to 23, wherein the poly-N-acetylglucosamine fibers have a β-1→4 configuration.

25. The method of any one of claims 1 to 24, wherein the sNAG nanofibers were produced from a microalgal poly-N-acetylglucosamine.

26. The method of any one of claims 1 to 25, wherein the sNAG nanofibers are formulated as a suspension or a gel.

27. The method of any one of claims 1 to 26, wherein the composition does not comprise an additional active ingredient.

28. The method of any one of claims 1 to 27, wherein the composition is injected into the gingiva of the subject using a syringe.

29. The method of any one of claims 1 to 28, wherein the composition comprises 5 to 30 mg of sNAG nanofibers per ml of an isotonic solution.

30. The method of claim 29, wherein the isotonic solution is a saline solution.

31. The method of claim 29 or 30, wherein approximately 30 to 40 microliters of the composition are injected into the gingiva of the subject.

32. The method of any one of claims 1 to 31, wherein the composition is injected into the coronal portion of the gingiva.

33. The method of any one of claims 1 to 32, wherein the composition is administered in conjunction with an anti-bacterial agent.

34. The method of claim 33, wherein the anti-bacterial agent is an antibiotic.

35. The method of any one of claims 1 to 34, wherein the composition is administered in conjunction with a pain reliever.

36. The method of any one of claims 1 to 34, wherein the composition is administered in conjunction with an anesthetic.

37. The method of any one of claims 1 to 32, wherein the composition is not administered in conjunction with an antibiotic.

38. The method of any one of claims 1 to 32, wherein the composition is not administered in conjunction with another therapy.

39. The method of any one of claims 1 to 38, wherein the injection of the composition into the gingiva of the subject is repeated on 2 to 6 separate occasions.

40. The method of any one of claims 1 to 39, wherein the injection of the composition into the gingiva improves the cement-enamel junction measurement at the mesial, mid or distal section of the coronal gingiva.

41. The method of any one of claims 1 to 40, wherein the injection of the composition decreases the reduction in the cement-enamel junction measurement at the mesial, mid or distal section of the coronal gingiva.

42. The method of any one of claims 1 to 41, wherein the injection of the composition decreases the reduction in the cement-enamel junction measurement at the mesial, mid and distal section of the coronal gingiva.

43. The method of any one of claims 1 to 42, wherein the injection of the composition reduces the thinning of the gingiva.

44. The method of any one of claims 1 to 43, wherein the subject is a human subject.

45. The method of claim 44, wherein the human subject is a human adult.

46. The method of claim 44, wherein the human subject is an elderly human.
